(19) United States
(12) Patent Application Publication
(10) Pub. No.: US 2025/0228891 A1
(43) Pub. Date: Jul. 17, 2025

(54) COMBINATIONS CONTAINING OXYLANTHANUM CARBONATE FOR TREATING HYPERPHOSPHATEMIA

(71) Applicant: UNICYCIVE THERAPEUTICS, INC., Los Altos, CA (US)

(72) Inventors: Satyanarayana Medicherla, Cupertino, CA (US); Pramod Kumar Gupta, Las Vegas, NV (US); Guru Reddy, Danville, CA (US); Shalabh K. Gupta, Los Altos, CA (US)

(21) Appl. No.: 19/017,192

(22) Filed: Jan. 10, 2025

Related U.S. Application Data
(60) Provisional application No. 63/620,727, filed on Jan. 12, 2024.

(51) Int. Cl. A61K 33/244; A61K 9/00; A61K 31/4725; A61P 13/12

(52) U.S. Cl. CPC A61K 33/244; A61K 9/0053; A61K 31/4725; A61P 13/12

(57) ABSTRACT

Combinations of oxylanthanum carbonate and tenapanor for reducing serum phosphate levels in patients with end-stage renal disease (ESRD) and/or chronic kidney disease (CKD).

### BACKGROUND

End-stage renal disease (ESRD) affects more than 7 million people worldwide and ˜70% of patients with ESRD have hyperphosphatemia. A cornerstone of hyperphosphatemia management in clinical practice is reduction of phosphate absorption from food through a combination of dietary phosphate restrictions and administration of oral phosphate binders. While restricting dietary phosphate intake can somewhat alleviate hyperphosphatemia, adherence to such restrictions is often challenging, particularly in view of the widespread use of high-phosphate additives in processed foods.

Phosphate binders work by physically sequestering dietary phosphate, making it unavailable for absorption, and effectively lowering serum phosphate levels. However, despite the use of phosphate binders, many ESRD patients struggle to maintain target serum phosphate levels, partially due to poor adherence to optimal phosphate binder administration protocols, which typically involve administration of a high volume of phosphate binders, often administered as large pills. Additionally, some phosphate binders can only be administered as powders or chewable pills, further adding to the complexity of the administration regimen.

Inadequate control of serum phosphate levels has substantial clinical implications, as elevated concentrations are linked to adverse patient outcomes, including increased all-cause mortality, cardiovascular events, left ventricular hypertrophy, and progression of chronic kidney disease (CKD) in patients not yet on dialysis.

Oxylanthanum carbonate (OLC) is a novel nanotechnology product that combines lanthanum, which has the highest binding capacity vs other phosphate binders, with a potentially smaller pill size that is swallowed with water vs chewed. Oxylanthanum carbonate with a high pore volume (over 0.015 cm³/g), methods of preparing same and pharmaceutical compositions thereof are described in U.S. Pat. Nos. 10,350,240, 11,406,663 and 8,961,917 whose contents are incorporated herein by reference in their entirety.

Tenapanor is a small-molecule inhibitor of the sodium/hydrogen exchanger isoform 3 (NHE3). NHE3 is expressed on the luminal surface of the small intestine and proximal colon and functions as a transporter to import luminal sodium in exchange for a cellular proton. Tenapanor is not a phosphate binder—instead, it decreases serum phosphate levels by locally inhibiting intestinal phosphate absorption. Tenapanor is currently FDA approved at a recommended dose of 30 mg twice daily (BID) to reduce serum phosphorus in adults with chronic kidney disease (CKD) on dialysis as add-on therapy in patients who have an inadequate response to phosphate binders or who are intolerant of any dose of phosphate binder therapy. In clinical trials, diarrhea was observed in 53% of patients raking tenapanor.

There is an unmet need for new combination regimens that can improve serum phosphate control in chronic kidney disease (CKD) and end-stage renal disease (ESRD) patients, reduce the incidence and severity of adverse events, in particular diarrhea related to tenapanor-containing regimens, increase treatment convenience by decreasing pill burden or providing more convenient administration options, or a combination thereof.

### SUMMARY OF INVENTION

In some embodiments provided is a method of reducing serum phosphate in a patient with end-stage renal disease (ESRD) in need thereof, the method comprising orally administering to the patient an oxylanthanum carbonate (OLC) and tenapanor.

In some embodiments provided is a method of reducing serum phosphate in a patient with diabetic or non-diabetic chronic kidney disease (CKD) in need thereof, the method comprising orally administering to the patient an oxylanthanum carbonate (OLC) and tenapanor. In some embodiments provided is a method of treating hyperphosphatemia in a patient in need thereof, the method comprising orally administering to the patient an oxylanthanum carbonate (OLC) and tenapanor.

In some embodiments provided is a method of treating hyperphosphatemia in a patient with diabetic or non-diabetic chronic kidney disease (CKD) in need thereof, the method comprising orally administering to the patient an oxylanthanum carbonate (OLC) and tenapanor.

In some embodiments provided is a method of reducing the incidence and/or severity of side effects associated with administration of phosphorus-lowering treatment in a patient suffering from ESRD in need of phosphorus-lowering treatment comprising orally administering to the patient an oxylanthanum carbonate (OLC) and tenapanor.

In some embodiments provided is a method of reducing the incidence and/or severity of side effects associated with administration of phosphorus-lowering treatment in a patient suffering from diabetic or non-diabetic chronic kidney disease (CKD) in need of phosphorus-lowering treatment comprising orally administering to the patient an oxylanthanum carbonate (OLC) and tenapanor.

In some embodiments provided is a method of reducing the incidence and/or severity of diarrhea associated with administration of tenapanor in a patient suffering from ESRD in need of tenapanor treatment comprising orally administering to the patient an oxylanthanum carbonate (OLC) and tenapanor.

In some embodiments provided is a method of reducing the incidence and/or severity of diarrhea associated with administration of tenapanor in a patient suffering from diabetic or non-diabetic CKD in need of tenapanor treatment comprising orally administering to the patient an oxylanthanum carbonate (OLC) and tenapanor.

In some embodiments provided is a method of reducing the incidence and/or severity of side effects associated with administration of lanthanum-containing phosphorus-reducing treatment in a patient suffering from ESRD in need of treatment with a lanthanum-containing phosphorus-reducing treatment comprising orally administering to the patient an oxylanthanum carbonate (OLC) and tenapanor.

In some embodiments provided is a method of reducing the incidence and/or severity of side effects associated with administration of lanthanum-containing phosphorus-reducing treatment in a patient suffering from diabetic or non-diabetic CKD in need of treatment with a lanthanum-containing phosphorus-reducing treatment comprising orally administering to the patient an oxylanthanum carbonate (OLC) and tenapanor.

In some embodiments provided is a method of reducing the incidence and/or severity of side effects associated with administration of phosphorus-lowering treatment in a patient suffering from ESRD in need of phosphorus-lowering treatment comprising orally administering to the patient an oxylanthanum carbonate (OLC) and tenapanor.

In some embodiments provided is a method of reducing the incidence and/or severity of side effects associated with administration of phosphorus-lowering treatment in a patient suffering from diabetic or non-diabetic CKD in need of phosphorus-lowering treatment comprising orally administering to the patient an oxylanthanum carbonate (OLC) and tenapanor.

In some embodiments provided is a method of reducing the incidence and/or severity of diarrhea associated with administration of tenapanor in a patient suffering from ESRD in need of tenapanor treatment comprising orally administering to the patient an oxylanthanum carbonate (OLC) and tenapanor.

In some embodiments provided is a method of reducing the incidence and/or severity of diarrhea associated with administration of tenapanor in a patient suffering from diabetic or non-diabetic CKD in need of tenapanor treatment comprising orally administering to the patient an oxylanthanum carbonate (OLC) and tenapanor.

In some embodiments provided is a method of reducing the incidence and/or severity of side effects associated with administration of lanthanum-containing phosphorus-reducing treatment in a patient suffering from ESRD in need of treatment with a lanthanum-containing phosphorus-reducing treatment comprising orally administering to the patient an oxylanthanum carbonate (OLC) and tenapanor.

In some embodiments provided is a method of reducing the incidence and/or severity of side effects associated with administration of lanthanum-containing phosphorus-reducing treatment in a patient suffering from diabetic or non-diabetic CKD in need of treatment with a lanthanum-containing phosphorus-reducing treatment comprising orally administering to the patient an oxylanthanum carbonate (OLC) and tenapanor.

In some embodiments, the tenapanor and the OLC are administered in synergistically effective amounts.

In some embodiments provided is a dosage form for oral administration comprising an effective amount of an oxylanthanum carbonate (OLC) and tenapanor.

In some embodiments, the dosage form is a swallowable tablet or caplet.

In some embodiments, the amount of OLC and the amount of tenapanor present in the dosage form are synergistically effective.

In some embodiments provided is a method of reducing serum phosphate in a patient with end-stage renal disease (ESRD) in need thereof, the method comprising orally administering to the patient a dosage form as defined in any of the embodiments described herein.

In some embodiments provided is a method of reducing serum phosphate in a patient with diabetic or non-diabetic chronic kidney disease (CKD) in need thereof, the method comprising orally administering to the patient a dosage form as defined in any of the embodiments described herein. In some embodiments provided is a method of treating hyperphosphatemia in a patient in need thereof, the method comprising orally administering to the patient a dosage form as defined in any of the embodiments described herein.

In some embodiments provided is a method of treating hyperphosphatemia in a patient with diabetic or non-diabetic chronic kidney disease (CKD) in need thereof, the method comprising orally administering to the patient a dosage form as defined in any of the embodiments described herein.

In some embodiments provided is a method of reducing the incidence and/or severity of side effects associated with administration of phosphorus-lowering treatment in a patient suffering from ESRD in need of phosphorus-lowering treatment comprising orally administering to the patient a dosage form as defined in any of the embodiments described herein.

In some embodiments provided is a method of reducing the incidence and/or severity of side effects associated with administration of phosphorus-lowering treatment in a patient suffering from diabetic or non-diabetic chronic kidney disease (CKD) in need of phosphorus-lowering treatment comprising orally administering to the patient a dosage form as defined in any of the embodiments described herein.

In some embodiments provided is a method of reducing the incidence and/or severity of diarrhea associated with administration of tenapanor in a patient suffering from ESRD in need of tenapanor treatment comprising orally administering to the patient a dosage form as defined in any of the embodiments described herein.

In some embodiments provided is a method of reducing the incidence and/or severity of diarrhea associated with administration of tenapanor in a patient suffering from diabetic or non-diabetic CKD in need of tenapanor treatment comprising orally administering to the patient a dosage form as defined in any of the embodiments described herein.

In some embodiments provided is a method of reducing the incidence and/or severity of side effects associated with administration of lanthanum-containing phosphorus-reducing treatment in a patient suffering from ESRD in need of treatment with a lanthanum-containing phosphorus-reducing treatment comprising orally administering to the patient a dosage form as defined in any of the embodiments described herein.

In some embodiments provided is a method of reducing the incidence and/or severity of side effects associated with administration of lanthanum-containing phosphorus-reducing treatment in a patient suffering from diabetic or non-diabetic CKD in need of treatment with a lanthanum-containing phosphorus-reducing treatment comprising orally administering to the patient a dosage form as defined in any of the embodiments described herein.

In some embodiments provided is a method of reducing the incidence and/or severity of side effects associated with administration of phosphorus-lowering treatment in a patient suffering from ESRD in need of phosphorus-lowering treatment comprising orally administering to the patient a dosage form as defined in any of the embodiments described herein.

In some embodiments provided is a method of reducing the incidence and/or severity of side effects associated with administration of phosphorus-lowering treatment in a patient suffering from diabetic or non-diabetic CKD in need of phosphorus-lowering treatment comprising orally administering to the patient a dosage form as defined in any of the embodiments described herein.

In some embodiments provided is a method of reducing the incidence and/or severity of diarrhea associated with administration of tenapanor in a patient suffering from ESRD in need of tenapanor treatment comprising orally administering to the patient a dosage form as defined in any of the embodiments described herein.

In some embodiments provided is a method of reducing the incidence and/or severity of diarrhea associated with administration of tenapanor in a patient suffering from diabetic or non-diabetic CKD in need of tenapanor treatment comprising orally administering to the patient a dosage form as defined in any of the embodiments described herein.

In some embodiments provided is a method of reducing the incidence and/or severity of side effects associated with administration of lanthanum-containing phosphorus-reducing treatment in a patient suffering from ESRD in need of treatment with a lanthanum-containing phosphorus-reducing treatment comprising orally administering to the patient a dosage form as defined in any of the embodiments described herein.

In some embodiments provided is a method of reducing the incidence and/or severity of side effects associated with administration of lanthanum-containing phosphorus-reducing treatment in a patient suffering from diabetic or non-diabetic CKD in need of treatment with a lanthanum-containing phosphorus-reducing treatment comprising orally administering to the patient a dosage form as defined in any of the embodiments described herein.

### BRIEF DESCRIPTION OF THE FIGURES

FIG. 1. Represents the Study Design of the OLC+Tenapanor Combo Study described in Example 1. The study consisted of acclimatization, randomization, in vivo study period, and termination. Animals were acclimated from Day-7 to Day-4. On study Day-1, animals were randomized into eight groups: one vehicle; one tenapanor (0.15 mg/kg); three oxylanthanum carbonate (OLC) (0.75%, 1.5%, and 3%); and three combination treatment with OLC and tenapanor (OLC 0.75%, 1.5%, and 3%+tenapanor 0.15 mg/kg). On study Day 1, animals were switched to high phosphorus diets. Animals were euthanized on Day 11 two hours post last dose. Legend: 1 Body weight and food intake were measured daily from study Day-3 until termination. 2 During the in vivo study period (Days 1 to 11), vehicle and tenapanor were dosed orally twice/day whereas OLC was incorporated into the diets. 3 Urine samples were collected on Days 9, 10, and 11 for phosphate measurements and blood samples were collected for serum phosphate and sodium measurements on day 11 after the final urine sampling.

FIGS. 2A and 2B represent the body weight (FIG. 2A) and food intake (FIG. 2B) by treatment group, OLC, Tenapanor, or combination treatment compared with vehicle group for the experiment described in Example 1. Legend: 1 Tenapanor dose=0.15 mg/kg/po/bid

FIG. 3A and FIG. 3B represent the daily mean (FIG. 3A) and pooled (FIG. 3B)+SE urinary phosphate excretion for days 9-11 of the study described in Example 1. OLC dose dependently decreased urinary phosphate excretion by treatment groups, (n=8 per Treatment Group), from days 9 to 11. When used together, OLC+tenapanor decreased urinary phosphate excretion such that combination reductions were larger compared to vehicle, OLC only, and tenapanor only. Legend: 1 Tenapanor dose 0.15 mg/kg/po/bid. 2 Average of all OLC doses (0.75%, 1.5%, 3%) in chow.

FIG. 4A represents the mean (+95% CI) urinary phosphate excretion by treatment groups, (n=8 per Treatment Group), from days 8 to 11. The tenapanor 0.15 mg/kg group, mean urinary phosphate excretion from Days 9 to 11 was 8.5 mg/day lower compared to the vehicle group. Legend. 1 Mean of urinary phosphate excretion levels for each rat from Days 9 to 11 was averaged by treatment group. 2 Tenapanor dose=0.15 mg/kg/po/bid 3 Statistical comparisons were conducted using one-way ANOVA with Dunnett's Multiple Comparison Test. FIG. 4B. represents the pooled (+95% CI) urinary phosphate excretion by treatment groups, (n=8 per Treatment Group), from days 8 to 11. The tenapanor 0.15 mg/kg group, mean urinary phosphate excretion from Days 9 to 11 was 8.5 mg/day lower compared to the vehicle group. In the oxylanthanum carbonate (OLC) alone groups, mean urinary phosphate excretion (pooled across the 0.75, 1.5, and 3% OLC dose groups) was 12 mg/day lower compared to the vehicle group. In the OLC+tenapanor groups (pooled across the 0.75, 1.5, and 3% OLC dose groups), urinary phosphate excretion was 28 mg/day lower compared to the vehicle group. OLC+tenapanor significantly decreased urinary phosphate excretion compared to OLC only and tenapanor only. Legend: 1 Mean urinary phosphate excretion levels for each rat from Days 9 to 11 was averaged by treatment group. 2 Tenapanor dose=0.15 mg/kg/po/bid. 3 Average of all OLC doses (0.75%, 1.5%, 3%) in chow. 4 Statistical comparisons were conducted using one-way ANOVA with Games-Howell Test.

FIG. 5. represents the mean percentage reduction of urinary phosphate excretion relative to vehicle group by treatment group from Days 9 to 11. The observed reduction in urinary phosphate excretion for the OLC+tenapanor groups was larger than the predicted reduction based on the single-agent effects in accordance with the Bliss model. The difference in observed (38%) vs. predicted (16%) urinary phosphate excretion was most pronounced for the combination group with the lowest OLC dose (0.75%). Legend: 1 Calculated by averaging urinary phosphate excretion levels for each rat from Days 9 to 11 by dose group then subtracting mean of urinary phosphate excretion levels from Days 9 to 11 of Vehicle 2 Tenapanor dose=0.15 mg/kg. 3 Predicted based on the Bliss Model; the model compares the predicted combination response calculated using the complete additivity of probability theory based on the observed effect of each individual agent administered alone, with the observed effect of combination treatment.

FIG. 6 represents the XRPD spectrum of the OLC of used in the formulations, dosage forms and methods of the invention.

### DETAILED DESCRIPTION

### Definitions

All percentages and ratios used herein are by weight of the total composition and all measurements made are at 25° C. and normal pressure unless otherwise designated. All temperatures are in Degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Preferably, such additives will not be present at all or only in trace amounts.

All ranges recited herein include the endpoints, including those that recite a range “between” two values. Terms such as “about,” “generally,” “substantially,” and the like are to be construed as modifying a term or value such that it is not an absolute, but does not read on the prior art, unless otherwise defined herein. Such terms will be defined by the circumstances and the terms that they modify as those terms are understood by those of skill in the art. This includes, at very least, the degree of expected experimental error, technique error and instrument error for a given technique used to measure a value.

Note that while the specification and claims may refer to a final product such as, for example, a tablet or other dosage form of the invention as, for example, containing particles having a certain particle size or distribution, or a certain type of, for example, a specific form of a filler, it may be difficult to tell from the final dosage form that the recitation is satisfied. However, such a recitation may be satisfied if the materials used prior to final production (in the case of a tablet for example, blending and tablet formulation), for example, meet that recitation. Indeed, as to any property or characteristic of a final product which cannot be ascertained from the dosage form directly, it is sufficient if that property resides in the components recited just prior to final production steps.

Where this document refers to a material, such as in this instance, oxylanthanum carbonate (OLC), by reference to patterns, spectra, or other graphical data, it may do so by qualifying that they are “substantially” as shown or depicted in a figure, or by one or more data points. By “substantially” used in such a context, it will be appreciated that patterns, spectra, and other graphical data can be shifted in their positions, relative intensities, or other values due to a number of factors known to those of skill in the art. For example, in the crystallographic and powder X-ray diffraction arts, shifts in peak positions or the relative intensities of one or more peaks of a pattern can occur because of, without limitation: the equipment used, the sample preparation protocol, preferred packing and orientations, the radiation source, operator error, method and length of data collection, and the like. However, those of ordinary skill in the art should be able to compare the figures herein with a pattern generated of an unknown and confirm its identity as one of the forms disclosed and claimed herein. The same holds true for other techniques which may be reported herein.

“Pharmaceutically acceptable” means approved or approvable by a regulatory agency of the Federal or state government or listed in the U.S. Pharmacopoeia or other generally recognized pharmacopoeia for use in animals, and more particularly in humans. It can be material which is not biologically or otherwise undesirable, i.e., the material can be administered to an individual without causing any undesirable biological effects or interacting in a deleterious manner with any of the components of the composition in which it is contained.

The term “pharmaceutically acceptable salt” of a compound means a salt that is pharmaceutically acceptable and that possesses the desired pharmacological activity of the parent compound. Such salts include, for example, acid addition salts and base addition salts.

“Acid addition salts” according to the present disclosure, are formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

“Base addition salts” according to the present disclosure are formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature can cause a single crystal form to dominate.

The term “treating” includes the administration of the compounds or agents of as provided for herein to a subject to prevent or delay, to alleviate, or to arrest or inhibit development of a disease or disorder, elevated serum phosphorus levels and hyperphosphatemia, in particular as associated with end-stage renal disease (ESRD) and chronic kidney disease (CKD). The term “treating” as used herein includes achieving a therapeutic benefit and/or a prophylactic benefit. By therapeutic benefit is meant eradication/normalization or amelioration/improvement/reversal/regression or stabilization/lack of progression of elevated phosphate levels or one or more of the physiological symptoms associated with elevated phosphate levels.

As used herein, and unless otherwise specified, a “therapeutically effective amount” of a compound is an amount sufficient alone or in combination with other therapies, to provide a therapeutic benefit in the therapeutic treatment of the disease, disorder or condition or to delay or minimize one or more symptoms associated with the disease, disorder or condition. The term “therapeutically effective amount” can encompass an amount that improves overall therapy, reduces or avoids symptoms or causes of disease or condition, or enhances the therapeutic efficacy of another therapeutic agent. In some embodiments, the “term “therapeutically effective amount” refers to a synergistically effective amount or synergistically therapeutic amount.

“Synergistic” means that the therapeutic effect of the OLC when administered in combination as described herein with a NHE3 inhibitor (e.g., tenapanor) is greater than the predicted additive therapeutic effects of the OLC and NHE3 inhibitor (e.g., tenapanor) when administered alone. The term “synergistically therapeutic amount” or “synergistically effective amount” refers to a less than standard therapeutic amount of one or both drugs, meaning that the amount required for the desired effect is lower than when the drug is used alone. A synergistically therapeutic amount also includes cases where one drug is given at a standard therapeutic dose and another drug is administered in a less than standard therapeutic dose. For example, the OLC could be given in a standard therapeutic dose and the NHE3 inhibitor (e.g., tenapanor) could be given in a less than standard therapeutic dose to provide a synergistic result, or vice versa. A synergistic effect can also be obtained when both drugs are given at their standard therapeutic doses, but the effect, for example the reduction of serum phosphate levels, is greater than the predicted additive effect of the two agents when administered alone at the same dose.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds as provided herein can be administered. In one aspect, the subject is a human subject. In some embodiments, methods are provided to identify subject patients for treatment according to the methods provided herein, accepted screening methods are employed to determine risk factors associated with a targeted or suspected disease or condition or to determine the status of an existing disease or condition in a subject. These screening methods include, for example, conventional work-ups to determine risk factors that are associated with the targeted or suspected disease or condition. These and other methods allow the clinician to select patients in need of therapy using the methods, compounds, compositions, and formulations as provided for herein.

### NHE3 Inhibitors

In some embodiments, the compositions and methods described herein contemplate combinations of OLC with NHE3 inhibitors. In some embodiments, the NHE3 inhibitors are substantially systemically non-bioavailable NHE3 inhibitors.

In some embodiments, the NHE3 inhibitor is tenapanor or a pharmaceutically acceptable salt thereof. In some embodiments, the compositions and methods described herein contemplate combinations of OLC with tenapanor or a pharmaceutically acceptable salt thereof. In some embodiments, the compositions and methods described herein contemplate combinations of OLC with tenapanor hydrochloride.

Tenapanor is approved by the FDA for reducing serum phosphorus in adults with chronic kidney disease (CKD) on dialysis as add-on therapy in patients who have an inadequate response to phosphate binders or who are intolerant of any dose of phosphate binder therapy and is marketed for this indication under the brand name XPHOZAH®. The recommended dose of tenapanor is 30 mg twice daily (BID).

The chemical name for tenapanor hydrochloride is 12,15-Dioxa-2,7,9-triazaheptadecanamide, 17-[[[3-[(4S)-6,8-dichloro-1,2,3,4-tetrahydro-2-methyl-4-isoquinolinyl]phenyl]sulphonyl]amino]-N-[2-[2-[2-[[[3-[(4S)-6,8-dichloro-1,2,3,4-tetrahydro-2-methyl-4-isoquinolinyl]phenyl]sulphonyl]amino]ethoxy]ethoxy]ethyl]-8-oxo-, hydrochloride (1:2). Tenapanor hydrochloride has the molecular formula of C₅₀H₆₈Cl₆N₈O₁₀S₂, the molecular weight of 1218 Daltons, and the chemical structure below:

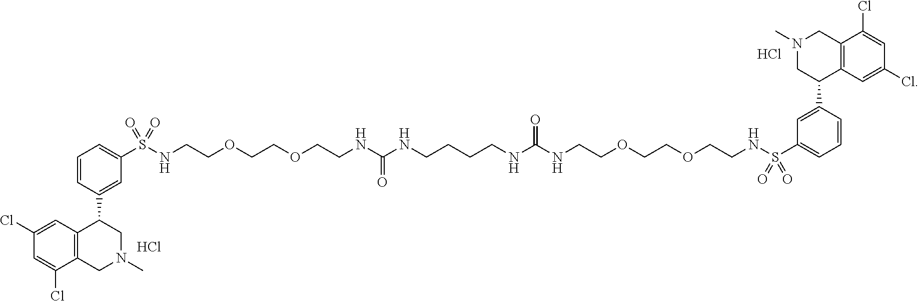 [SMILES: CN1Cc2c(Cl)cc(Cl)cc2[C@H](c2cccc(S(=O)(=O)NCCOCCOCCNC(=O)NCCCCNC(=O)NCCOCCOCCNS(=O)(=O)c3cccc([C@@H]4CN(C)Cc5c(Cl)cc(Cl)cc54)c3)c2)C1 ; Cl ; Cl]

References to amounts or doses of tenapanor refer to the amount of tenapanor free-base equivalent. For example, a dose of “10 mg tenapanor” can be provided as 10.6 mg. tenapanor hydrochloride, a dose of “20 mg tenapanor” can be provided as 21.3 mg. tenapanor hydrochloride and a dose of “30 mg tenapanor” can be provided as 31.9 mg. tenapanor hydrochloride.

Throughout the specification, references to administration of “tenapanor” include administration of tenapanor as a free base or as a pharmaceutically acceptable salt. References to dosage forms containing “tenapanor” include dosage forms containing tenapanor as a free base and tenapanor in the form of a pharmaceutically acceptable salt. In one embodiment, the tenapanor is in the form of tenapanor hydrochloride.

In some embodiments of the methods and compositions described herein, the dose of tenapanor is the recommended dose approved by the FDA for reducing serum phosphorus in adults with chronic kidney disease (CKD) on dialysis as add-on therapy in patients who have an inadequate response to phosphate binders or who are intolerant of any dose of phosphate binder therapy. In some embodiments, the dose of tenapanor is less than the recommended dose approved by the FDA for the above indication (e.g., 10% less, 20% less, 30% less, 40% less, 50% less, 60% less, 70% less, 75% less, 80% less, 90% less, 95% less, 96% less, 97% less, 98% less, 99% less).

In some embodiments of the compositions and methods described herein, the dose of tenapanor is between 0.1 mg and 30 mg. In some embodiments of the compositions and methods described herein, the dose of tenapanor is between 0.3 mg and 30 mg. In some embodiments of the compositions and methods described herein, the dose of tenapanor is between 0.5 mg and 30 mg. In some embodiments of the compositions and methods described herein, the dose of tenapanor is between 1 mg and 30 mg. In some embodiments of the compositions and methods described herein, the dose of tenapanor is between 5 mg and 30 mg. In some embodiments of the compositions and methods described herein, the dose of tenapanor is between 10 mg and 30 mg. In some embodiments of the compositions and methods described herein, the dose of tenapanor is between 15 mg and 30 mg. In some embodiments of the compositions and methods described herein, the dose of tenapanor is between 20 mg and 30 mg.

In some embodiments of the compositions and methods described herein, the dose of tenapanor is between 0.1 mg and 25 mg. In some embodiments of the compositions and methods described herein, the dose of tenapanor is between 0.3 mg and 25 mg. In some embodiments of the compositions and methods described herein, the dose of tenapanor is between 0.5 mg and 25 mg. In some embodiments of the compositions and methods described herein, the dose of tenapanor is between 1 mg and 25 mg. In some embodiments of the compositions and methods described herein, the dose of tenapanor is between 5 mg and 25 mg. In some embodiments of the compositions and methods described herein, the dose of tenapanor is between 10 mg and 25 mg. In some embodiments of the compositions and methods described herein, the dose of tenapanor is between 15 mg and 25 mg.

In some embodiments of the compositions and methods described herein, the dose of tenapanor is between 0.1 mg and 20 mg. In some embodiments of the compositions and methods described herein, the dose of tenapanor is between 0.3 mg and 20 mg. In some embodiments of the compositions and methods described herein, the dose of tenapanor is between 0.5 mg and 20 mg. In some embodiments of the compositions and methods described herein, the dose of tenapanor is between 1 mg and 20 mg. In some embodiments of the compositions and methods described herein, the dose of tenapanor is between 5 mg and 20 mg. In some embodiments of the compositions and methods described herein, the dose of tenapanor is between 10 mg and 20 mg. In some embodiments of the compositions and methods described herein, the dose of tenapanor is between 15 mg and 20 mg.

In some embodiments of the compositions and methods described herein, the dose of tenapanor is between 0.1 mg and 15 mg. In some embodiments of the compositions and methods described herein, the dose of tenapanor is between 0.3 mg and 15 mg. In some embodiments of the compositions and methods described herein, the dose of tenapanor is between 0.5 mg and 15 mg. In some embodiments of the compositions and methods described herein, the dose of tenapanor is between 1 mg and 15 mg. In some embodiments of the compositions and methods described herein, the dose of tenapanor is between 5 mg and 15 mg. In some embodiments of the compositions and methods described herein, the dose of tenapanor is between 10 mg and 15 mg.

In some embodiments of the compositions and methods described herein, the dose of tenapanor is between 0.1 mg and 10 mg. In some embodiments of the compositions and methods described herein, the dose of tenapanor is between 0.3 mg and 10 mg. In some embodiments of the compositions and methods described herein, the dose of tenapanor is between 0.5 mg and 10 mg. In some embodiments of the compositions and methods described herein, the dose of tenapanor is between 1 mg and 10 mg. In some embodiments of the compositions and methods described herein, the dose of tenapanor is between 5 mg and 10 mg.

In some embodiments of the compositions and methods described herein, the dose of tenapanor is between 0.1 mg and 5 mg. In some embodiments of the compositions and methods described herein, the dose of tenapanor is between 0.3 mg and 5 mg. In some embodiments of the compositions and methods described herein, the dose of tenapanor is between 0.5 mg and 5 mg. In some embodiments of the compositions and methods described herein, the dose of tenapanor is between 1 mg and 5 mg.

In some embodiments of the compositions and methods described herein, the dose of tenapanor is between 0.1 mg and 4 mg. In some embodiments of the compositions and methods described herein, the dose of tenapanor is between 0.3 mg and 4 mg. In some embodiments of the compositions and methods described herein, the dose of tenapanor is between 0.5 mg and 4 mg.

In some embodiments of the compositions and methods described herein, the dose of tenapanor is between 0.1 mg and 3 mg. In some embodiments of the compositions and methods described herein, the dose of tenapanor is between 0.3 mg and 3 mg. In some embodiments of the compositions and methods described herein, the dose of tenapanor is between 0.5 mg and 3 mg.

In some embodiments of the compositions and methods described herein, the dose of tenapanor is between 0.1 mg and 2 mg. In some embodiments of the compositions and methods described herein, the dose of tenapanor is between 0.3 mg and 2 mg. In some embodiments of the compositions and methods described herein, the dose of tenapanor is between 0.5 mg and 2 mg.

In some embodiments of the compositions and methods described herein, the dose of tenapanor is between 0.1 mg and 1 mg. In some embodiments of the compositions and methods described herein, the dose of tenapanor is between 0.3 mg and 1 mg. In some embodiments of the compositions and methods described herein, the dose of tenapanor is between 0.5 mg and 1 mg.

In some embodiments of the compositions or methods described herein, the dose of tenapanor is 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 10 mg, 15 mg, 20 mg, 25 mg or 30 mg. In some embodiments of the compositions or methods described herein, the dose of tenapanor is 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 10 mg, 15 mg, 20 mg or 25 mg. In some embodiments of the compositions or methods described herein, the dose of tenapanor is 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 10 mg, 15 mg, or 20 mg. In some embodiments of the compositions or methods described herein, the dose of tenapanor is 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 10 mg, or 15 mg. In some embodiments of the compositions or methods described herein, the dose of tenapanor is 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, or 10 mg. In some embodiments of the compositions or methods described herein, the dose of tenapanor is 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, or 5 mg. In some embodiments of the compositions or methods described herein, the dose of tenapanor is 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, or 4 mg. In some embodiments of the compositions or methods described herein, the dose of tenapanor is 0.1 mg, 0.2 mg, 0.3 mg, 0.4 mg, 0.5 mg, 0.6 mg, 0.7 mg, 0.8 mg, 0.9 mg, 1 mg, 2 mg, or 3 mg. In some embodiments of the compositions or methods described herein, the dose of tenapanor is 0.1 mg, 0.2 mg, 0.3 mg, 0.4 mg, 0.5 mg, 0.6 mg, 0.7 mg, 0.8 mg, 0.9 mg, 1 mg, or 2 mg. In some embodiments of the compositions or methods described herein, the dose of tenapanor is 0.1 mg, 0.2 mg, 0.3 mg, 0.4 mg, 0.5 mg, 0.6 mg, 0.7 mg, 0.8 mg, 0.9 mg, or 1 mg. In some embodiments of the compositions or methods described herein, the dose of tenapanor is 0.1 mg, 0.2 mg, 0.3 mg, 0.4 mg, 0.5 mg, 0.6 mg, 0.7 mg, 0.8 mg, or 0.9 mg. In some embodiments of the compositions or methods described herein, the dose of tenapanor is 0.1 mg, 0.2 mg, 0.3 mg, 0.4 mg, 0.5 mg, 0.6 mg, 0.7 mg, or 0.8 mg. In some embodiments of the compositions or methods described herein, the dose of tenapanor is 0.1 mg, 0.2 mg, 0.3 mg, 0.4 mg, 0.5 mg, 0.6 mg, or 0.7 mg. In some embodiments of the compositions or methods described herein, the dose of tenapanor is 0.1 mg, 0.2 mg, 0.3 mg, 0.4 mg, 0.5 mg, or 0.6 mg. In some embodiments of the compositions or methods described herein, the dose of tenapanor is 0.1 mg, 0.2 mg, 0.3 mg, 0.4 mg, or 0.5 mg. In some embodiments of the compositions or methods described herein, the dose of tenapanor is 0.1 mg, 0.2 mg, 0.3 mg, or 0.4 mg. In some embodiments of the compositions or methods described herein, the dose of tenapanor is 0.1 mg, 0.2 mg, or 0.3 mg. In some embodiments of the compositions or methods described herein, the dose of tenapanor is 0.1 mg or 0.2 mg. In some embodiments of the compositions or methods described herein, the dose of tenapanor is 0.1 mg.

In some embodiments of the compositions or methods described herein, the dose of tenapanor is 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 10 mg, 15 mg, 20 mg, 25 mg or 30 mg. In some embodiments of the compositions or methods described herein, the dose of tenapanor is 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 10 mg, 15 mg, 20 mg, or 25 mg. In some embodiments of the compositions or methods described herein, the dose of tenapanor is 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 10 mg, 15 mg, or 20 mg. In some embodiments of the compositions or methods described herein, the dose of tenapanor is 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 10 mg, or 15 mg. In some embodiments of the compositions or methods described herein, the dose of tenapanor is 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, or 10 mg. In some embodiments of the compositions or methods described herein, the dose of tenapanor is 0.2 mg, 0.3 mg, 0.4 mg, 0.5 mg, 0.6 mg, 0.7 mg, 0.8 mg, 0.9 mg, 1 mg, 2 mg, 3 mg, 4 mg, or 5 mg. In some embodiments of the compositions or methods described herein, the dose of tenapanor is 0.2 mg, 0.3 mg, 0.4 mg, 0.5 mg, 0.6 mg, 0.7 mg, 0.8 mg, 0.9 mg, 1 mg, 2 mg, 3 mg, or 4 mg. In some embodiments of the compositions or methods described herein, the dose of tenapanor is 0.2 mg, 0.3 mg, 0.4 mg, 0.5 mg, 0.6 mg, 0.7 mg, 0.8 mg, 0.9 mg, 1 mg, 2 mg, or 3 mg. In some embodiments of the compositions or methods described herein, the dose of tenapanor is 0.2 mg, 0.3 mg, 0.4 mg, 0.5 mg, 0.6 mg, 0.7 mg, 0.8 mg, 0.9 mg, 1 mg, or 2 mg. In some embodiments of the compositions or methods described herein, the dose of tenapanor is 0.2 mg, 0.3 mg, 0.4 mg, 0.5 mg, 0.6 mg, 0.7 mg, 0.8 mg, 0.9 mg, or 1 mg. In some embodiments of the compositions or methods described herein, the dose of tenapanor is 0.2 mg, 0.3 mg, 0.4 mg, 0.5 mg, 0.6 mg, 0.7 mg, 0.8 mg, or 0.9 mg. In some embodiments of the compositions or methods described herein, the dose of tenapanor is 0.2 mg, 0.3 mg, 0.4 mg, 0.5 mg, 0.6 mg, 0.7 mg, or 0.8 mg. In some embodiments of the compositions or methods described herein, the dose of tenapanor is 0.2 mg, 0.3 mg, 0.4 mg, 0.5 mg, 0.6 mg, or 0.7 mg. In some embodiments of the compositions or methods described herein, the dose of tenapanor is 0.2 mg, 0.3 mg, 0.4 mg, 0.5 mg, or 0.6 mg. In some embodiments of the compositions or methods described herein, the dose of tenapanor is 0.2 mg, 0.3 mg, 0.4 mg, or 0.5 mg. In some embodiments of the compositions or methods described herein, the dose of tenapanor is 0.2 mg, 0.3 mg, or 0.4 mg. In some embodiments of the compositions or methods described herein, the dose of tenapanor is 0.2 mg or 0.3 mg. In some embodiments of the compositions or methods described herein, the dose of tenapanor is 0.2 mg.

In some embodiments of the compositions or methods described herein, the dose of tenapanor is 0.3 mg, 0.4 mg, 0.5 mg, 0.6 mg, 0.7 mg, 0.8 mg, 0.9 mg, 1 mg, 2 mg, 3 mg, 4 mg, 5 mg, 10 mg, 15 mg, 20 mg, 25 mg, or 30 mg. In some embodiments of the compositions or methods described herein, the dose of tenapanor is 0.3 mg, 0.4 mg, 0.5 mg, 0.6 mg, 0.7 mg, 0.8 mg, 0.9 mg, 1 mg, 2 mg, 3 mg, 4 mg, 5 mg, 10 mg, 15 mg, 20 mg, or 25 mg. In some embodiments of the compositions or methods described herein, the dose of tenapanor is 0.3 mg, 0.4 mg, 0.5 mg, 0.6 mg, 0.7 mg, 0.8 mg, 0.9 mg, 1 mg, 2 mg, 3 mg, 4 mg, 5 mg, 10 mg, 15 mg, or 20 mg. In some embodiments of the compositions or methods described herein, the dose of tenapanor is 0.3 mg, 0.4 mg, 0.5 mg, 0.6 mg, 0.7 mg, 0.8 mg, 0.9 mg, 1 mg, 2 mg, 3 mg, 4 mg, 5 mg, 10 mg, or 15 mg. In some embodiments of the compositions or methods described herein, the dose of tenapanor is 0.3 mg, 0.4 mg, 0.5 mg, 0.6 mg, 0.7 mg, 0.8 mg, 0.9 mg, 1 mg, 2 mg, 3 mg, 4 mg, 5 mg, or 10 mg. In some embodiments of the compositions or methods described herein, the dose of tenapanor is 0.3 mg, 0.4 mg, 0.5 mg, 0.6 mg, 0.7 mg, 0.8 mg, 0.9 mg, 1 mg, 2 mg, 3 mg, 4 mg, or 5 mg. In some embodiments of the compositions or methods described herein, the dose of tenapanor is 0.3 mg, 0.4 mg, 0.5 mg, 0.6 mg, 0.7 mg, 0.8 mg, 0.9 mg, 1 mg, 2 mg, 3 mg, or 4 mg. In some embodiments of the compositions or methods described herein, the dose of tenapanor is 0.3 mg, 0.4 mg, 0.5 mg, 0.6 mg, 0.7 mg, 0.8 mg, 0.9 mg, 1 mg, 2 mg, or 3 mg. In some embodiments of the compositions or methods described herein, the dose of tenapanor is 0.3 mg, 0.4 mg, 0.5 mg, 0.6 mg, 0.7 mg, 0.8 mg, 0.9 mg, 1 mg, or 2 mg. In some embodiments of the compositions or methods described herein, the dose of tenapanor is 0.3 mg, 0.4 mg, 0.5 mg, 0.6 mg, 0.7 mg, 0.8 mg, 0.9 mg, or 1 mg. In some embodiments of the compositions or methods described herein, the dose of tenapanor is 0.3 mg, 0.4 mg, 0.5 mg, 0.6 mg, 0.7 mg, 0.8 mg, or 0.9 mg. In some embodiments of the compositions or methods described herein, the dose of tenapanor is 0.3 mg, 0.4 mg, 0.5 mg, 0.6 mg, 0.7 mg, or 0.8 mg. In some embodiments of the compositions or methods described herein, the dose of tenapanor is 0.3 mg, 0.4 mg, 0.5 mg, 0.6 mg, or 0.7 mg. In some embodiments of the compositions or methods described herein, the dose of tenapanor is 0.3 mg, 0.4 mg, 0.5 mg, or 0.6 mg. In some embodiments of the compositions or methods described herein, the dose of tenapanor is 0.3 mg, 0.4 mg, or 0.5 mg. In some embodiments of the compositions or methods described herein, the dose of tenapanor is 0.3 mg or 0.4 mg. In some embodiments of the compositions or methods described herein, the dose of tenapanor is 0.3 mg.

In some embodiments of the compositions or methods described herein, the dose of tenapanor is 0.4 mg, 0.5 mg, 0.6 mg, 0.7 mg, 0.8 mg, 0.9 mg, 1 mg, 2 mg, 3 mg, 4 mg, 5 mg, 10 mg, 15 mg, 20 mg, 25 mg, or 30 mg. In some embodiments of the compositions or methods described herein, the dose of tenapanor is 0.4 mg, 0.5 mg, 0.6 mg, 0.7 mg, 0.8 mg, 0.9 mg, 1 mg, 2 mg, 3 mg, 4 mg, 5 mg, 10 mg, 15 mg, 20 mg, or 25 mg. In some embodiments of the compositions or methods described herein, the dose of tenapanor is 0.4 mg, 0.5 mg, 0.6 mg, 0.7 mg, 0.8 mg, 0.9 mg, 1 mg, 2 mg, 3 mg, 4 mg, 5 mg, 10 mg, 15 mg, or 20 mg. In some embodiments of the compositions or methods described herein, the dose of tenapanor is 0.4 mg, 0.5 mg, 0.6 mg, 0.7 mg, 0.8 mg, 0.9 mg, 1 mg, 2 mg, 3 mg, 4 mg, 5 mg, 10 mg, or 15 mg. In some embodiments of the compositions or methods described herein, the dose of tenapanor is 0.4 mg, 0.5 mg, 0.6 mg, 0.7 mg, 0.8 mg, 0.9 mg, 1 mg, 2 mg, 3 mg, 4 mg, 5 mg, or 10 mg. In some embodiments of the compositions or methods described herein, the dose of tenapanor is 0.4 mg, 0.5 mg, 0.6 mg, 0.7 mg, 0.8 mg, 0.9 mg, 1 mg, 2 mg, 3 mg, 4 mg, or 5 mg. In some embodiments of the compositions or methods described herein, the dose of tenapanor is 0.4 mg, 0.5 mg, 0.6 mg, 0.7 mg, 0.8 mg, 0.9 mg, 1 mg, 2 mg, 3 mg, or 4 mg. In some embodiments of the compositions or methods described herein, the dose of tenapanor is 0.4 mg, 0.5 mg, 0.6 mg, 0.7 mg, 0.8 mg, 0.9 mg, 1 mg, 2 mg, or 3 mg. In some embodiments of the compositions or methods described herein, the dose of tenapanor is 0.4 mg, 0.5 mg, 0.6 mg, 0.7 mg, 0.8 mg, 0.9 mg, 1 mg, or 2 mg. In some embodiments of the compositions or methods described herein, the dose of tenapanor is 0.4 mg, 0.5 mg, 0.6 mg, 0.7 mg, 0.8 mg, 0.9 mg, or 1 mg. In some embodiments of the compositions or methods described herein, the dose of tenapanor is 0.4 mg, 0.5 mg, 0.6 mg, 0.7 mg, 0.8 mg, or 0.9 mg. In some embodiments of the compositions or methods described herein, the dose of tenapanor is 0.4 mg, 0.5 mg, 0.6 mg, 0.7 mg, or 0.8 mg. In some embodiments of the compositions or methods described herein, the dose of tenapanor is 0.4 mg, 0.5 mg, 0.6 mg, or 0.7 mg. In some embodiments of the compositions or methods described herein, the dose of tenapanor is 0.4 mg, 0.5 mg, or 0.6 mg. In some embodiments of the compositions or methods described herein, the dose of tenapanor is 0.4 mg or 0.5 mg. In some embodiments of the compositions or methods described herein, the dose of tenapanor is 0.4 mg.

In some embodiments of the compositions or methods described herein, the dose of tenapanor is 0.5 mg, 0.6 mg, 0.7 mg, 0.8 mg, 0.9 mg, 1 mg, 2 mg, 3 mg, 4 mg, 5 mg, 10 mg, 15 mg, 20 mg, 25 mg, or 30 mg. In some embodiments of the compositions or methods described herein, the dose of tenapanor is 0.5 mg, 0.6 mg, 0.7 mg, 0.8 mg, 0.9 mg, 1 mg, 2 mg, 3 mg, 4 mg, 5 mg, 10 mg, 15 mg, 20 mg, or 25 mg. In some embodiments of the compositions or methods described herein, the dose of tenapanor is 0.5 mg, 0.6 mg, 0.7 mg, 0.8 mg, 0.9 mg, 1 mg, 2 mg, 3 mg, 4 mg, 5 mg, 10 mg, 15 mg, or 20 mg. In some embodiments of the compositions or methods described herein, the dose of tenapanor is 0.5 mg, 0.6 mg, 0.7 mg, 0.8 mg, 0.9 mg, 1 mg, 2 mg, 3 mg, 4 mg, 5 mg, 10 mg, or 15 mg. In some embodiments of the compositions or methods described herein, the dose of tenapanor is 0.5 mg, 0.6 mg, 0.7 mg, 0.8 mg, 0.9 mg, 1 mg, 2 mg, 3 mg, 4 mg, 5 mg, or 10 mg. In some embodiments of the compositions or methods described herein, the dose of tenapanor is 0.5 mg, 0.6 mg, 0.7 mg, 0.8 mg, 0.9 mg, 1 mg, 2 mg, 3 mg, 4 mg, or 5 mg. In some embodiments of the compositions or methods described herein, the dose of tenapanor is 0.5 mg, 0.6 mg, 0.7 mg, 0.8 mg, 0.9 mg, 1 mg, 2 mg, 3 mg, or 4 mg. In some embodiments of the compositions or methods described herein, the dose of tenapanor is 0.5 mg, 0.6 mg, 0.7 mg, 0.8 mg, 0.9 mg, 1 mg, 2 mg, or 3 mg. In some embodiments of the compositions or methods described herein, the dose of tenapanor is 0.5 mg, 0.6 mg, 0.7 mg, 0.8 mg, 0.9 mg, 1 mg, or 2 mg. In some embodiments of the compositions or methods described herein, the dose of tenapanor is 0.5 mg, 0.6 mg, 0.7 mg, 0.8 mg, 0.9 mg, or 1 mg. In some embodiments of the compositions or methods described herein, the dose of tenapanor is 0.5 mg, 0.6 mg, 0.7 mg, 0.8 mg, or 0.9 mg. In some embodiments of the compositions or methods described herein, the dose of tenapanor is 0.5 mg, 0.6 mg, 0.7 mg, or 0.8 mg. In some embodiments of the compositions or methods described herein, the dose of tenapanor is 0.5 mg, 0.6 mg, or 0.7 mg. In some embodiments of the compositions or methods described herein, the dose of tenapanor is 0.5 mg or 0.6 mg. In some embodiments of the compositions or methods described herein, the dose of tenapanor is 0.5 mg.

In some embodiments of the compositions or methods described herein, the dose of tenapanor is 0.6 mg, 0.7 mg, 0.8 mg, 0.9 mg, 1 mg, 2 mg, 3 mg, 4 mg, 5 mg, 10 mg, 15 mg, 20 mg, 25 mg, or 30 mg. In some embodiments of the compositions or methods described herein, the dose of tenapanor is 0.6 mg, 0.7 mg, 0.8 mg, 0.9 mg, 1 mg, 2 mg, 3 mg, 4 mg, 5 mg, 10 mg, 15 mg, 20 mg, or 25 mg. In some embodiments of the compositions or methods described herein, the dose of tenapanor is 0.6 mg, 0.7 mg, 0.8 mg, 0.9 mg, 1 mg, 2 mg, 3 mg, 4 mg, 5 mg, 10 mg, 15 mg, or 20 mg. In some embodiments of the compositions or methods described herein, the dose of tenapanor is 0.6 mg, 0.7 mg, 0.8 mg, 0.9 mg, 1 mg, 2 mg, 3 mg, 4 mg, 5 mg, 10 mg, or 15 mg. In some embodiments of the compositions or methods described herein, the dose of tenapanor is 0.6 mg, 0.7 mg, 0.8 mg, 0.9 mg, 1 mg, 2 mg, 3 mg, 4 mg, 5 mg, or 10 mg. In some embodiments of the compositions or methods described herein, the dose of tenapanor is 0.6 mg, 0.7 mg, 0.8 mg, 0.9 mg, 1 mg, 2 mg, 3 mg, 4 mg, or 5 mg. In some embodiments of the compositions or methods described herein, the dose of tenapanor is 0.6 mg, 0.7 mg, 0.8 mg, 0.9 mg, 1 mg, 2 mg, 3 mg, or 4 mg. In some embodiments of the compositions or methods described herein, the dose of tenapanor is 0.6 mg, 0.7 mg, 0.8 mg, 0.9 mg, 1 mg, 2 mg, or 3 mg. In some embodiments of the compositions or methods described herein, the dose of tenapanor is 0.6 mg, 0.7 mg, 0.8 mg, 0.9 mg, 1 mg, or 2 mg. In some embodiments of the compositions or methods described herein, the dose of tenapanor is 0.6 mg, 0.7 mg, 0.8 mg, 0.9 mg, or 1 mg. In some embodiments of the compositions or methods described herein, the dose of tenapanor is 0.6 mg, 0.7 mg, 0.8 mg, or 0.9 mg. In some embodiments of the compositions or methods described herein, the dose of tenapanor is 0.6 mg, 0.7 mg, or 0.8 mg. In some embodiments of the compositions or methods described herein, the dose of tenapanor is 0.6 mg or 0.7 mg. In some embodiments of the compositions or methods described herein, the dose of tenapanor is 0.6 mg.

In some embodiments of the compositions or methods described herein, the dose of tenapanor is 0.7 mg, 0.8 mg, 0.9 mg, 1 mg, 2 mg, 3 mg, 4 mg, 5 mg, 10 mg, 15 mg, 20 mg, 25 mg, or 30 mg. In some embodiments of the compositions or methods described herein, the dose of tenapanor is 0.7 mg, 0.8 mg, 0.9 mg, 1 mg, 2 mg, 3 mg, 4 mg, 5 mg, 10 mg, 15 mg, 20 mg, or 25 mg. In some embodiments of the compositions or methods described herein, the dose of tenapanor is 0.7 mg, 0.8 mg, 0.9 mg, 1 mg, 2 mg, 3 mg, 4 mg, 5 mg, 10 mg, 15 mg, or 20 mg. In some embodiments of the compositions or methods described herein, the dose of tenapanor is 0.7 mg, 0.8 mg, 0.9 mg, 1 mg, 2 mg, 3 mg, 4 mg, 5 mg, 10 mg, or 15 mg. In some embodiments of the compositions or methods described herein, the dose of tenapanor is 0.7 mg, 0.8 mg, 0.9 mg, 1 mg, 2 mg, 3 mg, 4 mg, 5 mg, or 10 mg. In some embodiments of the compositions or methods described herein, the dose of tenapanor is 0.7 mg, 0.8 mg, 0.9 mg, 1 mg, 2 mg, 3 mg, 4 mg, or 5 mg. In some embodiments of the compositions or methods described herein, the dose of tenapanor is 0.7 mg, 0.8 mg, 0.9 mg, 1 mg, 2 mg, 3 mg, or 4 mg. In some embodiments of the compositions or methods described herein, the dose of tenapanor is 0.7 mg, 0.8 mg, 0.9 mg, 1 mg, 2 mg, or 3 mg. In some embodiments of the compositions or methods described herein, the dose of tenapanor is 0.7 mg, 0.8 mg, 0.9 mg, 1 mg, or 2 mg. In some embodiments of the compositions or methods described herein, the dose of tenapanor is 0.7 mg, 0.8 mg, 0.9 mg, or 1 mg. In some embodiments of the compositions or methods described herein, the dose of tenapanor is 0.7 mg, 0.8 mg, or 0.9 mg. In some embodiments of the compositions or methods described herein, the dose of tenapanor is 0.7 mg or 0.8 mg. In some embodiments of the compositions or methods described herein, the dose of tenapanor is 0.7 mg.

In some embodiments of the compositions or methods described herein, the dose of tenapanor is 0.8 mg, 0.9 mg, 1 mg, 2 mg, 3 mg, 4 mg, 5 mg, 10 mg, 15 mg, 20 mg, 25 mg, or 30 mg. In some embodiments of the compositions or methods described herein, the dose of tenapanor is 0.8 mg, 0.9 mg, 1 mg, 2 mg, 3 mg, 4 mg, 5 mg, 10 mg, 15 mg, 20 mg, or 25 mg. In some embodiments of the compositions or methods described herein, the dose of tenapanor is 0.8 mg, 0.9 mg, 1 mg, 2 mg, 3 mg, 4 mg, 5 mg, 10 mg, 15 mg, or 20 mg. In some embodiments of the compositions or methods described herein, the dose of tenapanor is 0.8 mg, 0.9 mg, 1 mg, 2 mg, 3 mg, 4 mg, 5 mg, 10 mg, or 15 mg. In some embodiments of the compositions or methods described herein, the dose of tenapanor is 0.8 mg, 0.9 mg, 1 mg, 2 mg, 3 mg, 4 mg, 5 mg, or 10 mg. In some embodiments of the compositions or methods described herein, the dose of tenapanor is 0.8 mg, 0.9 mg, 1 mg, 2 mg, 3 mg, 4 mg, or 5 mg. In some embodiments of the compositions or methods described herein, the dose of tenapanor is 0.8 mg, 0.9 mg, 1 mg, 2 mg, 3 mg, or 4 mg. In some embodiments of the compositions or methods described herein, the dose of tenapanor is 0.8 mg, 0.9 mg, 1 mg, 2 mg, or 3 mg. In some embodiments of the compositions or methods described herein, the dose of tenapanor is 0.8 mg, 0.9 mg, 1 mg, or 2 mg. In some embodiments of the compositions or methods described herein, the dose of tenapanor is 0.8 mg, 0.9 mg, or 1 mg. In some embodiments of the compositions or methods described herein, the dose of tenapanor is 0.8 mg or 0.9 mg. In some embodiments of the compositions or methods described herein, the dose of tenapanor is 0.8 mg.

In some embodiments of the compositions or methods described herein, the dose of tenapanor is 0.9 mg, 1 mg, 2 mg, 3 mg, 4 mg, 5 mg, 10 mg, 15 mg, 20 mg, 25 mg, or 30 mg. In some embodiments of the compositions or methods described herein, the dose of tenapanor is 0.9 mg, 1 mg, 2 mg, 3 mg, 4 mg, 5 mg, 10 mg, 15 mg, 20 mg, or 25 mg. In some embodiments of the compositions or methods described herein, the dose of tenapanor is 0.9 mg, 1 mg, 2 mg, 3 mg, 4 mg, 5 mg, 10 mg, 15 mg, or 20 mg. In some embodiments of the compositions or methods described herein, the dose of tenapanor is 0.9 mg, 1 mg, 2 mg, 3 mg, 4 mg, 5 mg, 10 mg, or 15 mg. In some embodiments of the compositions or methods described herein, the dose of tenapanor is 0.9 mg, 1 mg, 2 mg, 3 mg, 4 mg, 5 mg, or 10 mg. In some embodiments of the compositions or methods described herein, the dose of tenapanor is 0.9 mg, 1 mg, 2 mg, 3 mg, 4 mg, or 5 mg. In some embodiments of the compositions or methods described herein, the dose of tenapanor is 0.9 mg, 1 mg, 2 mg, 3 mg, or 4 mg. In some embodiments of the compositions or methods described herein, the dose of tenapanor is 0.9 mg, 1 mg, 2 mg, or 3 mg. In some embodiments of the compositions or methods described herein, the dose of tenapanor is 0.9 mg, 1 mg, or 2 mg. In some embodiments of the compositions or methods described herein, the dose of tenapanor is 0.9 mg or 1 mg. In some embodiments of the compositions or methods described herein, the dose of tenapanor is 0.9 mg.

In some embodiments of the compositions or methods described herein, the dose of tenapanor is 1 mg, 2 mg, 3 mg, 4 mg, 5 mg, 10 mg, 15 mg, 20 mg, 25 mg, or 30 mg. In some embodiments of the compositions or methods described herein, the dose of tenapanor is 1 mg, 2 mg, 3 mg, 4 mg, 5 mg, 10 mg, 15 mg, 20 mg, or 25 mg. In some embodiments of the compositions or methods described herein, the dose of tenapanor is 1 mg, 2 mg, 3 mg, 4 mg, 5 mg, 10 mg, 15 mg, or 20 mg. In some embodiments of the compositions or methods described herein, the dose of tenapanor is 1 mg, 2 mg, 3 mg, 4 mg, 5 mg, 10 mg, or 15 mg. In some embodiments of the compositions or methods described herein, the dose of tenapanor is 1 mg, 2 mg, 3 mg, 4 mg, 5 mg, or 10 mg. In some embodiments of the compositions or methods described herein, the dose of tenapanor is 1 mg, 2 mg, 3 mg, 4 mg, or 5 mg. In some embodiments of the compositions or methods described herein, the dose of tenapanor is 1 mg, 2 mg, 3 mg, or 4 mg. In some embodiments of the compositions or methods described herein, the dose of tenapanor is 1 mg, 2 mg, or 3 mg. In some embodiments of the compositions or methods described herein, the dose of tenapanor is 1 mg or 2 mg. In some embodiments of the compositions or methods described herein, the dose of tenapanor is 1 mg.

In some embodiments of the compositions or methods described herein, the dose of tenapanor is 2 mg, 3 mg, 4 mg, 5 mg, 10 mg, 15 mg, 20 mg, 25 mg, or 30 mg. In some embodiments of the compositions or methods described herein, the dose of tenapanor is 2 mg, 3 mg, 4 mg, 5 mg, 10 mg, 15 mg, 20 mg, or 25 mg. In some embodiments of the compositions or methods described herein, the dose of tenapanor is 2 mg, 3 mg, 4 mg, 5 mg, 10 mg, 15 mg, or 20 mg. In some embodiments of the compositions or methods described herein, the dose of tenapanor is 2 mg, 3 mg, 4 mg, 5 mg, 10 mg, or 15 mg. In some embodiments of the compositions or methods described herein, the dose of tenapanor is 2 mg, 3 mg, 4 mg, 5 mg, or 10 mg. In some embodiments of the compositions or methods described herein, the dose of tenapanor is 2 mg, 3 mg, 4 mg, or 5 mg. In some embodiments of the compositions or methods described herein, the dose of tenapanor is 2 mg, 3 mg, or 4 mg. In some embodiments of the compositions or methods described herein, the dose of tenapanor is 2 mg or 3 mg. In some embodiments of the compositions or methods described herein, the dose of tenapanor is 2 mg.

In some embodiments of the compositions or methods described herein, the dose of tenapanor is 3 mg, 4 mg, 5 mg, 10 mg, 15 mg, 20 mg, 25 mg, or 30 mg. In some embodiments of the compositions or methods described herein, the dose of tenapanor is 3 mg, 4 mg, 5 mg, 10 mg, 15 mg, 20 mg, or 25 mg. In some embodiments of the compositions or methods described herein, the dose of tenapanor is 3 mg, 4 mg, 5 mg, 10 mg, 15 mg, or 20 mg. In some embodiments of the compositions or methods described herein, the dose of tenapanor is 3 mg, 4 mg, 5 mg, 10 mg, or 15 mg. In some embodiments of the compositions or methods described herein, the dose of tenapanor is 3 mg, 4 mg, 5 mg, or 10 mg. In some embodiments of the compositions or methods described herein, the dose of tenapanor is 3 mg, 4 mg, or 5 mg. In some embodiments of the compositions or methods described herein, the dose of tenapanor is 3 mg or 4 mg. In some embodiments of the compositions or methods described herein, the dose of tenapanor is 3 mg.

In some embodiments of the compositions or methods described herein, the dose of tenapanor is 4 mg, 5 mg, 10 mg, 15 mg, 20 mg, 25 mg, or 30 mg. In some embodiments of the compositions or methods described herein, the dose of tenapanor is 4 mg, 5 mg, 10 mg, 15 mg, 20 mg, or 25 mg. In some embodiments of the compositions or methods described herein, the dose of tenapanor is 4 mg, 5 mg, 10 mg, 15 mg, or 20 mg. In some embodiments of the compositions or methods described herein, the dose of tenapanor is 4 mg, 5 mg, 10 mg, or 15 mg. In some embodiments of the compositions or methods described herein, the dose of tenapanor is 4 mg, 5 mg, or 10 mg. In some embodiments of the compositions or methods described herein, the dose of tenapanor is 4 mg or 5 mg. In some embodiments of the compositions or methods described herein, the dose of tenapanor is 4 mg.

In some embodiments of the compositions or methods described herein, the dose of tenapanor is 5 mg, 10 mg, 15 mg, 20 mg, 25 mg, or 30 mg. In some embodiments of the compositions or methods described herein, the dose of tenapanor is 5 mg, 10 mg, 15 mg, 20 mg, or 25 mg. In some embodiments of the compositions or methods described herein, the dose of tenapanor is 5 mg, 10 mg, 15 mg, or 20 mg. In some embodiments of the compositions or methods described herein, the dose of tenapanor is 5 mg, 10 mg, or 15 mg. In some embodiments of the compositions or methods described herein, the dose of tenapanor is 5 mg or 10 mg. In some embodiments of the compositions or methods described herein, the dose of tenapanor is 5 mg.

In some embodiments of the compositions or methods described herein, the dose of tenapanor is 10 mg, 15 mg, 20 mg, 25 mg, or 30 mg. In some embodiments of the compositions or methods described herein, the dose of tenapanor is 10 mg, 15 mg, 20 mg, or 25 mg. In some embodiments of the compositions or methods described herein, the dose of tenapanor is 10 mg, 15 mg, or 20 mg. In some embodiments of the compositions or methods described herein, the dose of tenapanor is 10 mg or 15 mg. In some embodiments of the compositions or methods described herein, the dose of tenapanor is 10 mg.

In some embodiments of the compositions or methods described herein, the dose of tenapanor is 15 mg, 20 mg, 25 mg, or 30 mg. In some embodiments of the compositions or methods described herein, the dose of tenapanor is 15 mg, 20 mg, or 25 mg. In some embodiments of the compositions or methods described herein, the dose of tenapanor is 15 mg or 20 mg. In some embodiments of the compositions or methods described herein, the dose of tenapanor is 15 mg.

In some embodiments of the compositions or methods described herein, the dose of tenapanor is 20 mg, 25 mg, or 30 mg. In some embodiments of the compositions or methods described herein, the dose of tenapanor is 20 mg or 25 mg. In some embodiments of the compositions or methods described herein, the dose of tenapanor is 20 mg.

In some embodiments of the compositions or methods described herein, the dose of tenapanor is 25 mg or 30 mg. In some embodiments of the compositions or methods described herein, the dose of tenapanor is 25 mg.

In some embodiments of the compositions or methods described herein, the dose of tenapanor is 30 mg.

In some embodiments of the compositions or methods described herein, the dose of tenapanor is 0.1 mg, 0.3 mg, 0.5 mg, 1 mg, 3 mg, 5 mg, 10 mg, 15 mg, 20 mg, 25 mg, or 30 mg. In some embodiments of the compositions or methods described herein, the dose of tenapanor is 0.1 mg, 0.3 mg, 0.5 mg, 1 mg, 3 mg, 5 mg, 10 mg, 15 mg, 20 mg, or 25 mg. In some embodiments of the compositions or methods described herein, the dose of tenapanor is 0.1 mg, 0.3 mg, 0.5 mg, 1 mg, 3 mg, 5 mg, 10 mg, 15 mg, or 20 mg. In some embodiments of the compositions or methods described herein, the dose of tenapanor is 0.1 mg, 0.3 mg, 0.5 mg, 1 mg, 3 mg, 5 mg, 10 mg, or 15 mg. In some embodiments of the compositions or methods described herein, the dose of tenapanor is 0.1 mg, 0.3 mg, 0.5 mg, 1 mg, 3 mg, 5 mg, or 10 mg. In some embodiments of the compositions or methods described herein, the dose of tenapanor is 0.1 mg, 0.3 mg, 0.5 mg, 1 mg, 3 mg, or 5 mg.

In some embodiments of the compositions or methods described herein, the dose of tenapanor is 1 mg, 3 mg, 5 mg, 10 mg, 15 mg, 20 mg, 25 mg, or 30 mg. In some embodiments of the compositions or methods described herein, the dose of tenapanor is 1 mg, 3 mg, 5 mg, 10 mg, 15 mg, 20 mg, or 25 mg. In some embodiments of the compositions or methods described herein, the dose of tenapanor is 1 mg, 3 mg, 5 mg, 10 mg, 15 mg, or 20 mg. In some embodiments of the compositions or methods described herein, the dose of tenapanor is 1 mg, 3 mg, 5 mg, 10 mg, or 15 mg. In some embodiments of the compositions or methods described herein, the dose of tenapanor is 1 mg, 3 mg, 5 mg, or 10 mg. In some embodiments of the compositions or methods described herein, the dose of tenapanor is 1 mg, 3 mg, or 5 mg.

In some embodiments of the compositions or methods described herein, the dose of tenapanor is 3 mg, 5 mg, 10 mg, 15 mg, 20 mg, 25 mg, or 30 mg. In some embodiments of the compositions or methods described herein, the dose of tenapanor is 3 mg, 5 mg, 10 mg, 15 mg, 20 mg, or 25 mg. In some embodiments of the compositions or methods described herein, the dose of tenapanor is 3 mg, 5 mg, 10 mg, 15 mg, or 20 mg. In some embodiments of the compositions or methods described herein, the dose of tenapanor is 3 mg, 5 mg, 10 mg, or 15 mg. In some embodiments of the compositions or methods described herein, the dose of tenapanor is 3 mg, 5 mg, or 10 mg. In some embodiments of the compositions or methods described herein, the dose of tenapanor is 3 mg or 5 mg.

In some embodiments of the methods described herein, the above doses of tenapanor are administered once daily. In some embodiments of the methods described herein, the above doses of tenapanor are administered twice daily. In some embodiments of the methods described herein, the above doses of tenapanor are administered thrice daily.

Each of the doses and dose ranges of tenapanor described herein can be administered in combination with each of the doses and dose ranges of OLC described herein.

### Oxylanthanum Carbonate (OLC)

The oxylanthanum carbonate (OLC) used in the compositions and methods described herein is the lanthanum oxycarbonate polymorph of formula La₂O₂CO₃ described in WO2011/143475 and U.S. Pat. Nos. 10,350,240, 11,406,663 and 8,961,917, incorporated herein by reference in their entirety. The oxylanthanum carbonate (OLC) used in the compositions and methods described herein has a pore volume of at least 0.015 cm³/g. In some embodiments, the OLC contains less than 0.75% by weight of sodium. In some embodiments, the oxylanthanum carbonate used in the compositions and methods described herein has a pore volume of at least 0.015 cm³/g and contains less than 0.75% by weight of sodium. In some embodiments, the OLC has a pore volume of at least 0.020 cm³/g. In some embodiments, the OLC has an average aggregate size (D50 by volume of aggregates measured by laser light-based techniques) of between about 4 and about 80 microns. In some embodiments, the OLC has a BET surface area of at least about 20 m²/g and often between about 30 and about 40 m²/g; a bulk density of about 0.1 to about 1.1 and in another embodiment, between about 0.5 and about 0.8 g/cc; an alkali-compound content of about 0.75% by weight (alkali-metal basis) or less.

In some embodiments, the OLC is produced by reacting a soluble salt of lanthanum chloride with a non-alkali carbonate (e.g., ammonium carbonate), and precipitating the product, resulting in a lanthanum carbonate hydroxide containing 0.5% by weight or less of sodium, followed by calcining the reaction product to provide the OLC with the formula La₂O₂CO₃, containing less than 0.75% by weight sodium. In some embodiments, the first step takes place in a solvent at a reaction temperature from between about 75 degrees C. to about 90 degrees C. at a pH of from about 6.0 to 7.5. In some embodiments, the calcining step takes place at a temperature between about 400 and about 700 degrees C. (e.g., about 550 degrees C.) for at least two hours.

Throughout the specification and claims, unless otherwise noted, references to amounts and doses of OLC refer to the amount of elemental lanthanum equivalent delivered by the OLC. For example, a “dose of 500 mg OLC” contains an amount of La₂O₂CO₃ that is equivalent to 500 mg elemental lanthanum (i.e., an amount of La₂O₂CO₃ that contains an amount of lanthanum equivalent to 500 mg elemental lanthanum).

The OLC used in the compositions and methods described herein has an XRPD pattern substantially corresponding to the pattern depicted in FIG. 8.

In some embodiments, the dose of OLC is a dose that is therapeutically effective as a single agent. In some embodiments, the dose of OLC is less than the dose that would be therapeutically effective as a single agent (e.g., 10% less, 20% less, 30% less, 40% less, 50% less, 60% less, 70% less, 75% less, 80% less, 90% less).

In some embodiments of the compositions and methods described herein, the dose of OLC is between 100 mg and 2000 mg. In some embodiments of the compositions and methods described herein, the dose of OLC is between 125 mg and 2000 mg. In some embodiments of the compositions and methods described herein, the dose of OLC is between 150 mg and 2000 mg. In some embodiments of the compositions and methods described herein, the dose of OLC is between 250 mg and 2000 mg. In some embodiments of the compositions and methods described herein, the dose of OLC is between 500 mg and 2000 mg. In some embodiments of the compositions and methods described herein, the dose of OLC is between 750 mg and 2000 mg. In some embodiments of the compositions and methods described herein, the dose of OLC is between 1000 mg and 2000 mg.

In some embodiments of the compositions and methods described herein, the dose of OLC is between 100 mg and 1500 mg. In some embodiments of the compositions and methods described herein, the dose of OLC is between 125 mg and 1500 mg. In some embodiments of the compositions and methods described herein, the dose of OLC is between 150 mg and 1500 mg. In some embodiments of the compositions and methods described herein, the dose of OLC is between 250 mg and 1500 mg. In some embodiments of the compositions and methods described herein, the dose of OLC is between 500 mg and 1500 mg. In some embodiments of the compositions and methods described herein, the dose of OLC is between 750 mg and 1500 mg. In some embodiments of the compositions and methods described herein, the dose of OLC is between 1000 mg and 1500 mg.

In some embodiments of the compositions and methods described herein, the dose of OLC is between 100 mg and 1250 mg. In some embodiments of the compositions and methods described herein, the dose of OLC is between 125 mg and 1250 mg. In some embodiments of the compositions and methods described herein, the dose of OLC is between 150 mg and 1250 mg. In some embodiments of the compositions and methods described herein, the dose of OLC is between 250 mg and 1250 mg. In some embodiments of the compositions and methods described herein, the dose of OLC is between 500 mg and 1250 mg. In some embodiments of the compositions and methods described herein, the dose of OLC is between 750 mg and 1250 mg. In some embodiments of the compositions and methods described herein, the dose of OLC is between 900 mg and 1250 mg.

In some embodiments of the compositions and methods described herein, the dose of OLC is between 100 mg and 1000 mg. In some embodiments of the compositions and methods described herein, the dose of OLC is between 125 mg and 1000 mg. In some embodiments of the compositions and methods described herein, the dose of OLC is between 150 mg and 1000 mg. In some embodiments of the compositions and methods described herein, the dose of OLC is between 250 mg and 1000 mg. In some embodiments of the compositions and methods described herein, the dose of OLC is between 500 mg and 1000 mg. In some embodiments of the compositions and methods described herein, the dose of OLC is between 750 mg and 1000 mg.

In some embodiments of the compositions and methods described herein, the dose of OLC is between 100 mg and 750 mg. In some embodiments of the compositions and methods described herein, the dose of OLC is between 125 mg and 750 mg. In some embodiments of the compositions and methods described herein, the dose of OLC is between 150 mg and 750 mg. In some embodiments of the compositions and methods described herein, the dose of OLC is between 250 mg and 750 mg. In some embodiments of the compositions and methods described herein, the dose of OLC is between 500 mg and 750 mg.

In some embodiments of the compositions and methods described herein, the dose of OLC is between 100 mg and 500 mg. In some embodiments of the compositions and methods described herein, the dose of OLC is between 125 mg and 500 mg. In some embodiments of the compositions and methods described herein, the dose of OLC is between 150 mg and 500 mg. In some embodiments of the compositions and methods described herein, the dose of OLC is between 250 mg and 500 mg.

In some embodiments of the compositions and methods described herein, the dose of OLC is between 100 mg and 250 mg. In some embodiments of the compositions and methods described herein, the dose of OLC is between 125 mg and 250 mg. In some embodiments of the compositions and methods described herein, the dose of OLC is between 150 mg and 250 mg. In some embodiments of the compositions and methods described herein, the dose of OLC is 100 mg, 125 mg, 150 mg, 200 mg, 250 mg, 300 mg, 350 mg, 400 mg, 450 mg, 500 mg, 600 mg, 700 mg, 750 mg, 800 mg, 900 mg, 1000 mg, 1250 mg, 1500 mg or 2000 mg. In some embodiments of the compositions and methods described herein, the dose of OLC is 100 mg, 125 mg, 150 mg, 200 mg, 250 mg, 300 mg, 350 mg, 400 mg, 450 mg, 500 mg, 600 mg, 700 mg, 750 mg, 800 mg, 900 mg, 1000 mg, 1250 mg or 1500 mg. In some embodiments of the compositions and methods described herein, the dose of OLC is 100 mg, 125 mg, 150 mg, 200 mg, 250 mg, 300 mg, 350 mg, 400 mg, 450 mg, 500 mg, 600 mg, 700 mg, 750 mg, 800 mg, 900 mg, 1000 mg, or 1250 mg. In some embodiments of the compositions and methods described herein, the dose of OLC is 100 mg, 125 mg, 150 mg, 200 mg, 250 mg, 300 mg, 350 mg, 400 mg, 450 mg, 500 mg, 600 mg, 700 mg, 750 mg, 800 mg, 900 mg, or 1000 mg. In some embodiments of the compositions and methods described herein, the dose of OLC is 100 mg, 125 mg, 150 mg, 200 mg, 250 mg, 300 mg, 350 mg, 400 mg, 450 mg, 500 mg, 600 mg, 700 mg, 750 mg, 800 mg, or 900 mg. In some embodiments of the compositions and methods described herein, the dose of OLC is 100 mg, 125 mg, 150 mg, 200 mg, 250 mg, 300 mg, 350 mg, 400 mg, 450 mg, 500 mg, 600 mg, 700 mg, 750 mg, or 800 mg. In some embodiments of the compositions and methods described herein, the dose of OLC is 100 mg, 125 mg, 150 mg, 200 mg, 250 mg, 300 mg, 350 mg, 400 mg, 450 mg, 500 mg, 600 mg, 700 mg, or 750 mg. In some embodiments of the compositions and methods described herein, the dose of OLC is 100 mg, 125 mg, 150 mg, 200 mg, 250 mg, 300 mg, 350 mg, 400 mg, 450 mg, 500 mg, 600 mg, or 700 mg. In some embodiments of the compositions and methods described herein, the dose of OLC is 100 mg, 125 mg, 150 mg, 200 mg, 250 mg, 300 mg, 350 mg, 400 mg, 450 mg, 500 mg, or 600 mg. In some embodiments of the compositions and methods described herein, the dose of OLC is 100 mg, 125 mg, 150 mg, 200 mg, 250 mg, 300 mg, 350 mg, 400 mg, 450 mg, or 500 mg. In some embodiments of the compositions and methods described herein, the dose of OLC is 100 mg, 125 mg, 150 mg, 200 mg, 250 mg, 300 mg, 350 mg, 400 mg, or 450 mg. In some embodiments of the compositions and methods described herein, the dose of OLC is 100 mg, 125 mg, 150 mg, 200 mg, 250 mg, 300 mg, 350 mg, or 400 mg. In some embodiments of the compositions and methods described herein, the dose of OLC is 100 mg, 125 mg, 150 mg, 200 mg, 250 mg, 300 mg, or 350 mg. In some embodiments of the compositions and methods described herein, the dose of OLC is 100 mg, 125 mg, 150 mg, 200 mg, 250 mg, or 300 mg. In some embodiments of the compositions and methods described herein, the dose of OLC is 100 mg, 125 mg, 150 mg, 200 mg, or 250 mg. In some embodiments of the compositions and methods described herein, the dose of OLC is 100 mg, 125 mg, 150 mg, or 200 mg. In some embodiments of the compositions and methods described herein, the dose of OLC is 100 mg, 125 mg, or 150 mg. In some embodiments of the compositions and methods described herein, the dose of OLC is 100 mg or 125 mg. In some embodiments of the compositions and methods described herein, the dose of OLC is 100 mg.

In some embodiments of the compositions and methods described herein, the dose of OLC is 125 mg, 150 mg, 200 mg, 250 mg, 300 mg, 350 mg, 400 mg, 450 mg, 500 mg, 600 mg, 700 mg, 750 mg, 800 mg, 900 mg, 1000 mg, 1250 mg, 1500 mg or 2000 mg. In some embodiments of the compositions and methods described herein, the dose of OLC is 125 mg, 150 mg, 200 mg, 250 mg, 300 mg, 350 mg, 400 mg, 450 mg, 500 mg, 600 mg, 700 mg, 750 mg, 800 mg, 900 mg, 1000 mg, 1250 mg, or 1500 mg. In some embodiments of the compositions and methods described herein, the dose of OLC is 125 mg, 150 mg, 200 mg, 250 mg, 300 mg, 350 mg, 400 mg, 450 mg, 500 mg, 600 mg, 700 mg, 750 mg, 800 mg, 900 mg, 1000 mg, or 1250 mg. In some embodiments of the compositions and methods described herein, the dose of OLC is 125 mg, 150 mg, 200 mg, 250 mg, 300 mg, 350 mg, 400 mg, 450 mg, 500 mg, 600 mg, 700 mg, 750 mg, 800 mg, 900 mg, or 1000 mg. In some embodiments of the compositions and methods described herein, the dose of OLC is 125 mg, 150 mg, 200 mg, 250 mg, 300 mg, 350 mg, 400 mg, 450 mg, 500 mg, 600 mg, 700 mg, 750 mg, 800 mg, or 900 mg. In some embodiments of the compositions and methods described herein, the dose of OLC is 125 mg, 150 mg, 200 mg, 250 mg, 300 mg, 350 mg, 400 mg, 450 mg, 500 mg, 600 mg, 700 mg, 750 mg, or 800 mg. In some embodiments of the compositions and methods described herein, the dose of OLC is 125 mg, 150 mg, 200 mg, 250 mg, 300 mg, 350 mg, 400 mg, 450 mg, 500 mg, 600 mg, 700 mg, or 750 mg. In some embodiments of the compositions and methods described herein, the dose of OLC is 125 mg, 150 mg, 200 mg, 250 mg, 300 mg, 350 mg, 400 mg, 450 mg, 500 mg, 600 mg, or 700 mg. In some embodiments of the compositions and methods described herein, the dose of OLC is 125 mg, 150 mg, 200 mg, 250 mg, 300 mg, 350 mg, 400 mg, 450 mg, 500 mg, or 600 mg. In some embodiments of the compositions and methods described herein, the dose of OLC is 125 mg, 150 mg, 200 mg, 250 mg, 300 mg, 350 mg, 400 mg, 450 mg, or 500 mg. In some embodiments of the compositions and methods described herein, the dose of OLC is 125 mg, 150 mg, 200 mg, 250 mg, 300 mg, 350 mg, 400 mg, or 450 mg. In some embodiments of the compositions and methods described herein, the dose of OLC is 125 mg, 150 mg, 200 mg, 250 mg, 300 mg, 350 mg, or 400 mg. In some embodiments of the compositions and methods described herein, the dose of OLC is 125 mg, 150 mg, 200 mg, 250 mg, 300 mg, or 350 mg. In some embodiments of the compositions and methods described herein, the dose of OLC is 125 mg, 150 mg, 200 mg, 250 mg, or 300 mg. In some embodiments of the compositions and methods described herein, the dose of OLC is 125 mg, 150 mg, 200 mg, or 250 mg. In some embodiments of the compositions and methods described herein, the dose of OLC is 125 mg, 150 mg, or 200 mg. In some embodiments of the compositions and methods described herein, the dose of OLC is 125 mg or 150 mg. In some embodiments of the compositions and methods described herein, the dose of OLC is 125 mg.

In some embodiments of the compositions and methods described herein, the dose of OLC is 150 mg, 125 mg, 200 mg, 250 mg, 300 mg, 350 mg, 400 mg, 450 mg, 500 mg, 600 mg, 700 mg, 750 mg, 800 mg, 900 mg, 1000 mg, 1250 mg, 1500 mg or 2000 mg. In some embodiments of the compositions and methods described herein, the dose of OLC is 150 mg, 200 mg, 250 mg, 300 mg, 350 mg, 400 mg, 450 mg, 500 mg, 600 mg, 700 mg, 800 mg, 900 mg, 1000 mg, 1250 mg, or 1500 mg. In some embodiments of the compositions and methods described herein, the dose of OLC is 150 mg, 200 mg, 250 mg, 300 mg, 350 mg, 400 mg, 450 mg, 500 mg, 600 mg, 700 mg, 800 mg, 900 mg, 1000 mg, or 1250 mg. In some embodiments of the compositions and methods described herein, the dose of OLC is 150 mg, 200 mg, 250 mg, 300 mg, 350 mg, 400 mg, 450 mg, 500 mg, 600 mg, 700 mg, 800 mg, 900 mg, or 1000 mg. In some embodiments of the compositions and methods described herein, the dose of OLC is 150 mg, 200 mg, 250 mg, 300 mg, 350 mg, 400 mg, 450 mg, 500 mg, 600 mg, 700 mg, 800 mg, or 900 mg. In some embodiments of the compositions and methods described herein, the dose of OLC is 150 mg, 200 mg, 250 mg, 300 mg, 350 mg, 400 mg, 450 mg, 500 mg, 600 mg, 700 mg, 800 mg. In some embodiments of the compositions and methods described herein, the dose of OLC is 150 mg, 200 mg, 250 mg, 300 mg, 350 mg, 400 mg, 450 mg, 500 mg, 600 mg, 700 mg or In some embodiments of the compositions and methods described herein, the dose of OLC is 150 mg, 200 mg, 250 mg, 300 mg, 350 mg, 400 mg, 450 mg, 500 mg, 600 mg or 700 mg. In some embodiments of the compositions and methods described herein, the dose of OLC is 150 mg, 200 mg, 250 mg, 300 mg, 350 mg, 400 mg, 450 mg, 500 mg, or 600 mg. In some embodiments of the compositions and methods described herein, the dose of OLC is 150 mg, 200 mg, 250 mg, 300 mg, 350 mg, 400 mg, 450 mg, or 500 mg. In some embodiments of the compositions and methods described herein, the dose of OLC is 150 mg, 200 mg, 250 mg, 300 mg, 350 mg, 400 mg, or 450 mg. In some embodiments of the compositions and methods described herein, the dose of OLC is 150 mg, 200 mg, 250 mg, 300 mg, 350 mg, or 400 mg. In some embodiments of the compositions and methods described herein, the dose of OLC is 150 mg, 200 mg, 250 mg, 300 mg, or 350 mg. In some embodiments of the compositions and methods described herein, the dose of OLC is 150 mg, 200 mg, 250 mg, or 300 mg. In some embodiments of the compositions and methods described herein, the dose of OLC is 150 mg, 200 mg, or 250 mg. In some embodiments of the compositions and methods described herein, the dose of OLC is 150 mg or 200 mg. In some embodiments of the compositions and methods described herein, the dose of OLC is 150 mg.

In some embodiments of the compositions and methods described herein, the dose of OLC is 200 mg, 250 mg, 300 mg, 350 mg, 400 mg, 450 mg, 500 mg, 600 mg, 700 mg, 800 mg, 900 mg, 1000 mg, 1250 mg, 1500 mg, or 2000 mg. In some embodiments of the compositions and methods described herein, the dose of OLC is 200 mg, 250 mg, 300 mg, 350 mg, 400 mg, 450 mg, 500 mg, 600 mg, 700 mg, 800 mg, 900 mg, 1000 mg, 1250 mg, or 1500 mg. In some embodiments of the compositions and methods described herein, the dose of OLC is 200 mg, 250 mg, 300 mg, 350 mg, 400 mg, 450 mg, 500 mg, 600 mg, 700 mg, 800 mg, 900 mg, 1000 mg, or 1250 mg. In some embodiments of the compositions and methods described herein, the dose of OLC is 200 mg, 250 mg, 300 mg, 350 mg, 400 mg, 450 mg, 500 mg, 600 mg, 700 mg, 800 mg, 900 mg, or 1000 mg. In some embodiments of the compositions and methods described herein, the dose of OLC is 200 mg, 250 mg, 300 mg, 350 mg, 400 mg, 450 mg, 500 mg, 600 mg, 700 mg, 800 mg, or 900 mg. In some embodiments of the compositions and methods described herein, the dose of OLC is 200 mg, 250 mg, 300 mg, 350 mg, 400 mg, 450 mg, 500 mg, 600 mg, 700 mg, 800 mg. In some embodiments of the compositions and methods described herein, the dose of OLC is 200 mg, 250 mg, 300 mg, 350 mg, 400 mg, 450 mg, 500 mg, 600 mg, 700 mg or In some embodiments of the compositions and methods described herein, the dose of OLC is 200 mg, 250 mg, 300 mg, 350 mg, 400 mg, 450 mg, 500 mg, 600 mg or 700 mg. In some embodiments of the compositions and methods described herein, the dose of OLC is 200 mg, 250 mg, 300 mg, 350 mg, 400 mg, 450 mg, 500 mg, or 600 mg. In some embodiments of the compositions and methods described herein, the dose of OLC is 200 mg, 250 mg, 300 mg, 350 mg, 400 mg, 450 mg, or 500 mg. In some embodiments of the compositions and methods described herein, the dose of OLC is 200 mg, 250 mg, 300 mg, 350 mg, 400 mg, or 450 mg. In some embodiments of the compositions and methods described herein, the dose of OLC is 200 mg, 250 mg, 300 mg, 350 mg, or 400 mg. In some embodiments of the compositions and methods described herein, the dose of OLC is 200 mg, 250 mg, 300 mg, or 350 mg. In some embodiments of the compositions and methods described herein, the dose of OLC is 200 mg, 250 mg, or 300 mg. In some embodiments of the compositions and methods described herein, the dose of OLC is 200 mg or 250 mg. In some embodiments of the compositions and methods described herein, the dose of OLC is 200 mg.

In some embodiments of the compositions and methods described herein, the dose of OLC is 250 mg, 300 mg, 350 mg, 400 mg, 450 mg, 500 mg, 600 mg, 700 mg, 750 mg, 800 mg, 900 mg, 1000 mg, 1250 mg, 1500 mg, or 2000 mg. In some embodiments of the compositions and methods described herein, the dose of OLC is 250 mg, 300 mg, 350 mg, 400 mg, 450 mg, 500 mg, 600 mg, 700 mg, 800 mg, 900 mg, 1000 mg, 1250 mg, or 1500 mg. In some embodiments of the compositions and methods described herein, the dose of OLC is 250 mg, 300 mg, 350 mg, 400 mg, 450 mg, 500 mg, 600 mg, 700 mg, 800 mg, 900 mg, 1000 mg, or 1250 mg. In some embodiments of the compositions and methods described herein, the dose of OLC is 250 mg, 300 mg, 350 mg, 400 mg, 450 mg, 500 mg, 600 mg, 700 mg, 800 mg, 900 mg, or 1000 mg. In some embodiments of the compositions and methods described herein, the dose of OLC is 250 mg, 300 mg, 350 mg, 400 mg, 450 mg, 500 mg, 600 mg, 700 mg, 800 mg, or 900 mg. In some embodiments of the compositions and methods described herein, the dose of OLC is 250 mg, 300 mg, 350 mg, 400 mg, 450 mg, 500 mg, 600 mg, 700 mg, 800 mg. In some embodiments of the compositions and methods described herein, the dose of OLC is 250 mg, 300 mg, 350 mg, 400 mg, 450 mg, 500 mg, 600 mg, 700 mg or In some embodiments of the compositions and methods described herein, the dose of OLC is 250 mg, 300 mg, 350 mg, 400 mg, 450 mg, 500 mg, 600 mg or 700 mg. In some embodiments of the compositions and methods described herein, the dose of OLC is 250 mg, 300 mg, 350 mg, 400 mg, 450 mg, 500 mg, or 600 mg. In some embodiments of the compositions and methods described herein, the dose of OLC is 250 mg, 300 mg, 350 mg, 400 mg, 450 mg, or 500 mg. In some embodiments of the compositions and methods described herein, the dose of OLC is 250 mg, 300 mg, 350 mg, 400 mg, or 450 mg. In some embodiments of the compositions and methods described herein, the dose of OLC is 250 mg, 300 mg, 350 mg, or 400 mg. In some embodiments of the compositions and methods described herein, the dose of OLC is 250 mg, 300 mg, or 350 mg. In some embodiments of the compositions and methods described herein, the dose of OLC is 250 mg or 300 mg. In some embodiments of the compositions and methods described herein, the dose of OLC is 250 mg.

In some embodiments of the compositions and methods described herein, the dose of OLC is 300 mg, 350 mg, 400 mg, 450 mg, 500 mg, 600 mg, 700 mg, 800 mg, 900 mg, 1000 mg, 1250 mg, 1500 mg, or 2000 mg. In some embodiments of the compositions and methods described herein, the dose of OLC is 300 mg, 350 mg, 400 mg, 450 mg, 500 mg, 600 mg, 700 mg, 800 mg, 900 mg, 1000 mg, 1250 mg, or 1500 mg. In some embodiments of the compositions and methods described herein, the dose of OLC is 300 mg, 350 mg, 400 mg, 450 mg, 500 mg, 600 mg, 700 mg, 800 mg, 900 mg, 1000 mg, or 1250 mg. In some embodiments of the compositions and methods described herein, the dose of OLC is 300 mg, 350 mg, 400 mg, 450 mg, 500 mg, 600 mg, 700 mg, 800 mg, 900 mg, or 1000 mg. In some embodiments of the compositions and methods described herein, the dose of OLC is 300 mg, 350 mg, 400 mg, 450 mg, 500 mg, 600 mg, 700 mg, 800 mg, or 900 mg. In some embodiments of the compositions and methods described herein, the dose of OLC is 300 mg, 350 mg, 400 mg, 450 mg, 500 mg, 600 mg, 700 mg, 800 mg. In some embodiments of the compositions and methods described herein, the dose of OLC is 300 mg, 350 mg, 400 mg, 450 mg, 500 mg, 600 mg, 700 mg or In some embodiments of the compositions and methods described herein, the dose of OLC is 300 mg, 350 mg, 400 mg, 450 mg, 500 mg, 600 mg or 700 mg. In some embodiments of the compositions and methods described herein, the dose of OLC is 300 mg, 350 mg, 400 mg, 450 mg, 500 mg, or 600 mg. In some embodiments of the compositions and methods described herein, the dose of OLC is 300 mg, 350 mg, 400 mg, 450 mg, or 500 mg. In some embodiments of the compositions and methods described herein, the dose of OLC is 300 mg, 350 mg, 400 mg, or 450 mg. In some embodiments of the compositions and methods described herein, the dose of OLC is 300 mg, 350 mg, or 400 mg. In some embodiments of the compositions and methods described herein, the dose of OLC is 300 mg or 350 mg. In some embodiments of the compositions and methods described herein, the dose of OLC is 300 mg.

In some embodiments of the compositions and methods described herein, the dose of OLC is 350 mg, 400 mg, 450 mg, 500 mg, 600 mg, 700 mg, 750 mg, 800 mg, 900 mg, 1000 mg, 1250 mg, 1500 mg, or 2000 mg. In some embodiments of the compositions and methods described herein, the dose of OLC is 350 mg, 400 mg, 450 mg, 500 mg, 600 mg, 700 mg, 750 mg, 800 mg, 900 mg, 1000 mg, 1250 mg, or 1500 mg. In some embodiments of the compositions and methods described herein, the dose of OLC is 350 mg, 400 mg, 450 mg, 500 mg, 600 mg, 700 mg, 750 mg, 800 mg, 900 mg, 1000 mg, or 1250 mg. In some embodiments of the compositions and methods described herein, the dose of OLC is 350 mg, 400 mg, 450 mg, 500 mg, 600 mg, 700 mg, 750 mg, 800 mg, 900 mg, or 1000 mg. In some embodiments of the compositions and methods described herein, the dose of OLC is 350 mg, 400 mg, 450 mg, 500 mg, 600 mg, 700 mg, 750 mg, 800 mg, or 900 mg. In some embodiments of the compositions and methods described herein, the dose of OLC is 350 mg, 400 mg, 450 mg, 500 mg, 600 mg, 700 mg, 750 mg, or 800 mg. In some embodiments of the compositions and methods described herein, the dose of OLC is 350 mg, 400 mg, 450 mg, 500 mg, 600 mg, 700 mg, or 750 mg. In some embodiments of the compositions and methods described herein, the dose of OLC is 350 mg, 400 mg, 450 mg, 500 mg, 600 mg, or 700 mg. In some embodiments of the compositions and methods described herein, the dose of OLC is 350 mg, 400 mg, 450 mg, 500 mg, or 600 mg. In some embodiments of the compositions and methods described herein, the dose of OLC is 350 mg, 400 mg, 450 mg, or 500 mg. In some embodiments of the compositions and methods described herein, the dose of OLC is 350 mg, 400 mg, or 450 mg. In some embodiments of the compositions and methods described herein, the dose of OLC is 350 mg or 400 mg. In some embodiments of the compositions and methods described herein, the dose of OLC is 350 mg.

In some embodiments of the compositions and methods described herein, the dose of OLC is 400 mg, 450 mg, 500 mg, 600 mg, 700 mg, 750 mg, 800 mg, 900 mg, 1000 mg, 1250 mg, 1500 mg, or 2000 mg. In some embodiments of the compositions and methods described herein, the dose of OLC is 400 mg, 450 mg, 500 mg, 600 mg, 700 mg, 750 mg, 800 mg, 900 mg, 1000 mg, 1250 mg, or 1500 mg. In some embodiments of the compositions and methods described herein, the dose of OLC is 400 mg, 450 mg, 500 mg, 600 mg, 700 mg, 750 mg, 800 mg, 900 mg, 1000 mg, or 1250 mg. In some embodiments of the compositions and methods described herein, the dose of OLC is 400 mg, 450 mg, 500 mg, 600 mg, 700 mg, 750 mg, 800 mg, 900 mg, or 1000 mg. In some embodiments of the compositions and methods described herein, the dose of OLC is 400 mg, 450 mg, 500 mg, 600 mg, 700 mg, 750 mg, 800 mg, or 900 mg. In some embodiments of the compositions and methods described herein, the dose of OLC is 400 mg, 450 mg, 500 mg, 600 mg, 700 mg, 750 mg, or 800 mg. In some embodiments of the compositions and methods described herein, the dose of OLC is 400 mg, 450 mg, 500 mg, 600 mg, 700 mg, or 750 mg. In some embodiments of the compositions and methods described herein, the dose of OLC is 400 mg, 450 mg, 500 mg, 600 mg, or 700 mg. In some embodiments of the compositions and methods described herein, the dose of OLC is 400 mg, 450 mg, 500 mg, or 600 mg. In some embodiments of the compositions and methods described herein, the dose of OLC is 400 mg, 450 mg, or 500 mg. In some embodiments of the compositions and methods described herein, the dose of OLC is 400 mg or 450 mg. In some embodiments of the compositions and methods described herein, the dose of OLC is 400 mg.

In some embodiments of the compositions and methods described herein, the dose of OLC is 450 mg, 500 mg, 600 mg, 700 mg, 750 mg, 800 mg, 900 mg, 1000 mg, 1250 mg, 1500 mg, or 2000 mg. In some embodiments of the compositions and methods described herein, the dose of OLC is 450 mg, 500 mg, 600 mg, 700 mg, 750 mg, 800 mg, 900 mg, 1000 mg, 1250 mg, or 1500 mg. In some embodiments of the compositions and methods described herein, the dose of OLC is 450 mg, 500 mg, 600 mg, 700 mg, 750 mg, 800 mg, 900 mg, 1000 mg, or 1250 mg. In some embodiments of the compositions and methods described herein, the dose of OLC is 450 mg, 500 mg, 600 mg, 700 mg, 750 mg, 800 mg, 900 mg, or 1000 mg. In some embodiments of the compositions and methods described herein, the dose of OLC is 450 mg, 500 mg, 600 mg, 700 mg, 750 mg, 800 mg, or 900 mg. In some embodiments of the compositions and methods described herein, the dose of OLC is 450 mg, 500 mg, 600 mg, 700 mg, 750 mg, or 800 mg. In some embodiments of the compositions and methods described herein, the dose of OLC is 450 mg, 500 mg, 600 mg, 700 mg, or 750 mg. In some embodiments of the compositions and methods described herein, the dose of OLC is 450 mg, 500 mg, 600 mg, or 700 mg. In some embodiments of the compositions and methods described herein, the dose of OLC is 450 mg, 500 mg, or 600 mg. In some embodiments of the compositions and methods described herein, the dose of OLC is 450 mg or 500 mg. In some embodiments of the compositions and methods described herein, the dose of OLC is 450 mg.

In some embodiments of the compositions and methods described herein, the dose of OLC is 500 mg, 600 mg, 700 mg, 750 mg, 800 mg, 900 mg, 1000 mg, 1250 mg, 1500 mg, or 2000 mg. In some embodiments of the compositions and methods described herein, the dose of OLC is 500 mg, 600 mg, 700 mg, 750 mg, 800 mg, 900 mg, 1000 mg, 1250 mg, or 1500 mg. In some embodiments of the compositions and methods described herein, the dose of OLC is 500 mg, 600 mg, 700 mg, 750 mg, 800 mg, 900 mg, 1000 mg, or 1250 mg. In some embodiments of the compositions and methods described herein, the dose of OLC is 500 mg, 600 mg, 700 mg, 750 mg, 800 mg, 900 mg, or 1000 mg. In some embodiments of the compositions and methods described herein, the dose of OLC is 500 mg, 600 mg, 700 mg, 750 mg, 800 mg, or 900 mg. In some embodiments of the compositions and methods described herein, the dose of OLC is 500 mg, 600 mg, 700 mg, 750 mg, or 800 mg. In some embodiments of the compositions and methods described herein, the dose of OLC is 500 mg, 600 mg, 700 mg, or 750 mg. In some embodiments of the compositions and methods described herein, the dose of OLC is 500 mg, 600 mg, or 700 mg. In some embodiments of the compositions and methods described herein, the dose of OLC is 500 mg or 600 mg. In some embodiments of the compositions and methods described herein, the dose of OLC is 500 mg.

In some embodiments of the compositions and methods described herein, the dose of OLC is 600 mg, 700 mg, 750 mg, 800 mg, 900 mg, 1000 mg, 1250 mg, 1500 mg, or 2000 mg. In some embodiments of the compositions and methods described herein, the dose of OLC is 600 mg, 700 mg, 750 mg, 800 mg, 900 mg, 1000 mg, 1250 mg, or 1500 mg. In some embodiments of the compositions and methods described herein, the dose of OLC is 600 mg, 700 mg, 750 mg, 800 mg, 900 mg, 1000 mg, or 1250 mg. In some embodiments of the compositions and methods described herein, the dose of OLC is 600 mg, 700 mg, 750 mg, 800 mg, 900 mg, or 1000 mg. In some embodiments of the compositions and methods described herein, the dose of OLC is 600 mg, 700 mg, 750 mg, 800 mg, or 900 mg. In some embodiments of the compositions and methods described herein, the dose of OLC is 600 mg, 700 mg, 750 mg, or 800 mg. In some embodiments of the compositions and methods described herein, the dose of OLC is 600 mg, 700 mg, or 750 mg. In some embodiments of the compositions and methods described herein, the dose of OLC is 600 mg or 700 mg. In some embodiments of the compositions and methods described herein, the dose of OLC is 600 mg.

In some embodiments of the compositions and methods described herein, the dose of OLC is 700 mg, 750 mg, 800 mg, 900 mg, 1000 mg, 1250 mg, 1500 mg, or 2000 mg. In some embodiments of the compositions and methods described herein, the dose of OLC is 700 mg, 750 mg, 800 mg, 900 mg, 1000 mg, 1250 mg, or 1500 mg. In some embodiments of the compositions and methods described herein, the dose of OLC is 700 mg, 750 mg, 800 mg, 900 mg, 1000 mg, or 1250 mg. In some embodiments of the compositions and methods described herein, the dose of OLC is 700 mg, 750 mg, 800 mg, 900 mg, or 1000 mg. In some embodiments of the compositions and methods described herein, the dose of OLC is 700 mg, 750 mg, 800 mg, or 900 mg. In some embodiments of the compositions and methods described herein, the dose of OLC is 700 mg, 750 mg, or 800 mg. In some embodiments of the compositions and methods described herein, the dose of OLC is 700 mg or 750 mg. In some embodiments of the compositions and methods described herein, the dose of OLC is 700 mg.

In some embodiments of the compositions and methods described herein, the dose of OLC is 750 mg, 800 mg, 900 mg, 1000 mg, 1250 mg, 1500 mg, or 2000 mg. In some embodiments of the compositions and methods described herein, the dose of OLC is 750 mg, 800 mg, 900 mg, 1000 mg, 1250 mg, or 1500 mg. In some embodiments of the compositions and methods described herein, the dose of OLC is 750 mg, 800 mg, 900 mg, 1000 mg, or 1250 mg. In some embodiments of the compositions and methods described herein, the dose of OLC is 750 mg, 800 mg, 900 mg, or 1000 mg. In some embodiments of the compositions and methods described herein, the dose of OLC is 750 mg, 800 mg, or 900 mg. In some embodiments of the compositions and methods described herein, the dose of OLC is 750 mg or 800 mg. In some embodiments of the compositions and methods described herein, the dose of OLC is 750 mg.

In some embodiments of the compositions and methods described herein, the dose of OLC is 800 mg, 900 mg, 1000 mg, 1250 mg, 1500 mg, or 2000 mg. In some embodiments of the compositions and methods described herein, the dose of OLC is 800 mg, 900 mg, 1000 mg, 1250 mg, or 1500 mg. In some embodiments of the compositions and methods described herein, the dose of OLC is 800 mg, 900 mg, 1000 mg, or 1250 mg. In some embodiments of the compositions and methods described herein, the dose of OLC is 800 mg, 900 mg, or 1000 mg. In some embodiments of the compositions and methods described herein, the dose of OLC is 800 mg or 900 mg. In some embodiments of the compositions and methods described herein, the dose of OLC is 800 mg.

In some embodiments of the compositions and methods described herein, the dose of OLC is 900 mg, 1000 mg, 1250 mg, 1500 mg, or 2000 mg. In some embodiments of the compositions and methods described herein, the dose of OLC is 900 mg, 1000 mg, 1250 mg, or 1500 mg. In some embodiments of the compositions and methods described herein, the dose of OLC is 900 mg, 1000 mg, or 1250 mg. In some embodiments of the compositions and methods described herein, the dose of OLC is 900 mg or 1000 mg. In some embodiments of the compositions and methods described herein, the dose of OLC is 900 mg.

In some embodiments of the compositions and methods described herein, the dose of OLC is 1000 mg, 1250 mg, 1500 mg, or 2000 mg. In some embodiments of the compositions and methods described herein, the dose of OLC is 1000 mg, 1250 mg, or 1500 mg. In some embodiments of the compositions and methods described herein, the dose of OLC is 1000 mg or 1250 mg. In some embodiments of the compositions and methods described herein, the dose of OLC is 1000 mg.

In some embodiments of the compositions and methods described herein, the dose of OLC is 1250 mg, 1500 mg, or 2000 mg. In some embodiments of the compositions and methods described herein, the dose of OLC is 1250 mg or 1500 mg. In some embodiments of the compositions and methods described herein, the dose of OLC is 1250 mg.

In some embodiments of the compositions and methods described herein, the dose of OLC is 1500 mg or 2000 mg. In some embodiments of the compositions and methods described herein, the dose of OLC is 1500 mg.

In some embodiments of the compositions and methods described herein, the dose of OLC is 2000 mg.

In some embodiments of the compositions and methods described herein, the dose of OLC is 100 mg, 125 mg, 250 mg, 500 mg, 750 mg, 1000 mg, 1500 mg, or 2000 mg. In some embodiments of the compositions and methods described herein, the dose of OLC is 100 mg, 125 mg, 250 mg, 500 mg, 750 mg, 1000 mg, 1250 mg, or 1500 mg. In some embodiments of the compositions and methods described herein, the dose of OLC is 100 mg, 125 mg, 250 mg, 500 mg, 750 mg, 1000 mg, or 1250 mg. In some embodiments of the compositions and methods described herein, the dose of OLC is 100 mg, 125 mg, 250 mg, 500 mg, 750 mg, or 1000 mg. In some embodiments of the compositions and methods described herein, the dose of OLC is 100 mg, 125 mg, 250 mg, 500 mg, or 750 mg. In some embodiments of the compositions and methods described herein, the dose of OLC is 100 mg, 125 mg, 250 mg, or 500 mg.

In some embodiments of the compositions and methods described herein, the dose of OLC is 125 mg, 250 mg, 500 mg, 750 mg, 1000 mg, 1500 mg, or 2000 mg. In some embodiments of the compositions and methods described herein, the dose of OLC is 125 mg, 250 mg, 500 mg, 750 mg, 1000 mg, 1250 mg, or 1500 mg. In some embodiments of the compositions and methods described herein, the dose of OLC is 125 mg, 250 mg, 500 mg, 750 mg, 1000 mg, or 1250 mg. In some embodiments of the compositions and methods described herein, the dose of OLC is 125 mg, 250 mg, 500 mg, 750 mg, or 1000 mg. In some embodiments of the compositions and methods described herein, the dose of OLC is 125 mg, 250 mg, 500 mg, or 750 mg. In some embodiments of the compositions and methods described herein, the dose of OLC is 125 mg, 250 mg, or 500 mg.

In some embodiments of the compositions and methods described herein, the dose of OLC is 500 mg, 750 mg, 1000 mg, 1500 mg, or 2000 mg. In some embodiments of the compositions and methods described herein, the dose of OLC is 250 mg, 500 mg, 750 mg, 1000 mg, 1250 mg, or 1500 mg. In some embodiments of the compositions and methods described herein, the dose of OLC is 250 mg, 500 mg, 750 mg, 1000 mg, or 1250 mg. In some embodiments of the compositions and methods described herein, the dose of OLC is 250 mg, 500 mg, 750 mg, or 1000 mg. In some embodiments of the compositions and methods described herein, the dose of OLC is 250 mg, 500 mg, or 750 mg. In some embodiments of the compositions and methods described herein, the dose of OLC is 250 mg or 500 mg.

In some embodiments of the methods described herein, the above doses of OLC are administered once daily. In some embodiments of the methods described herein, the above doses of OLC are administered twice daily. In some embodiments of the methods described herein, the above doses of OLC are administered thrice daily.

Each of the doses and dose ranges of OLC described herein can be administered in combination with each of the doses and dose ranges of tenapanor described herein.

### Pharmaceutical Compositions and Dosage Forms

Pharmaceutical compositions comprising an effective amount of the OLC and/or tenapanor and at least one pharmaceutically acceptable excipient are also contemplated.

In one aspect, provided are dosage forms comprising a pharmaceutical composition comprising OLC and/or tenapanor, wherein the dosage form is selected from the group consisting of swallow tablets, swallow caplets, compressed dosage forms, swallow hard gelatin capsules, swallow soft gel capsules, orally dissolvable tablets, orally dissolvable caplets, orally dissolvable hard gelatin capsules, orally dissolvable soft gelatin capsules, chewable tablets, chewable caplets, chewable capsules, powders, sprinkles, orally disintegrable films, foods, confections, gums, syrups, suspensions, emulsions or dispersions. In one embodiment, the dosage form is a swallow tablet or swallow caplet. In one embodiment, the dosage form is a powder for oral administration.

In some embodiment, the dosage forms comprise OLC and pharmaceutical excipients without tenapanor. In some embodiment, the dosage forms comprise OLC and tenapanor as a fixed dose combination. The fixed dose combination can contain any of the doses of OLC and tenapanor described herein in any combination.

It is contemplated that the dosage forms may also include at least one other ingredient or pharmaceutically acceptable excipient. This excipient may include, but is not limited to, taste masking agents, coatings, mass diluting agents, binders, fillers, sugars, sweeteners, including artificial sweeteners, polymers, flavoring agents, coloring agents, lubricants, glidants, bio- or muco-adhesives, viscosity modifiers, surfactants, buffers, disintegrant s, compression/encapsulation aids, plasticizers, slip/anti-electrostatic agents, etc. The amount of any one or more of these excipients will vary with, inter alia, the amount and type of API, API particle size, and shape of the dosage form, form of the dosage form, desired speed of release of active (e.g., within seconds or minutes after ingestion), desired location of release of active in the body, how many ingredients are used, which ingredients are used, the number of dosage forms that will make-up a dose, the amount of API(s) per dose and the like.

Taste masking agent(s) include anything known to be used as a taste masking agents in this art. Preferred taste masking agents may include Eudragit E-100, ethylcellulose, hydroxypropylmethylcellulose, hydroxypropyl cellulose, methylcellulose, hydroxyethylcellulose, carboxymethylcellulose, shellac, zein, carbomers, and fats. Taste masking agents can be used in conventional amounts and preferably in an amount of about 1 to about 5% by weight of the total dosage form, and more preferably in an amount of about 2% to about 5% by weight of the total dosage form, and most preferably in an amount of about 2% to about 3% by weight of the total dosage form.

Binders can be anything known to be used as binders. These materials are used to add cohesiveness to powders and provide the necessary bonding to form granules that can be compressed into hard tablets that have acceptable mechanical strength to withstand subsequent processing or shipping and handling. Some binders include acacia, tragacanth, gelatin, starch (both modified or unmodified), cellulose materials such as methylcellulose, ethylcellulose, hydroxypropylmethylcellulose, hydroxypropyl cellulose, hydroxyethylcellulose and sodium carboxy methylcellulose, alginic acids and salts thereof, e.g., sodium alginate, magnesium aluminum silicate, polyethylene glycol, guar gum, xanthan gum, polysaccharide acids, bentonites, sugars, invert sugars, and the like, fats, waxes, carbopol, povidone, polyvinylpyrrolidone, polymethacrylate and other acrylic and vinyl-based polymers. Binders can be used in conventional amounts and preferably in an amount of about 0 by weight to about 50 and more preferably about 2 to about 10 percent by weight of the total dosage form.

Coating agents, where included, are typically present in a trace amount by weight. Nonlimiting examples of coating agents include cellulose phthalate, cellulose acetate phthalate, ethylcellulose, gellan gum, maltodextrin, methacrylates, methylcellulose, microcrystalline cellulose and carrageenan, shellac, sucrose, and polyvinyl derivatives. Where a coating is used, it may be added, for example, to slow the disintegration of the tablet after administration (e.g., polymer coating) or to extend shelf life by shielding the tablet from picking up moisture. Fillers can be anything known to be used as fillers. Some fillers include mannitol, dextrose, sorbitol, lactose, sucrose, and calcium carbonate. Fillers can be used in conventional amounts and preferably in an amount of about 0 to about 90, and more preferably about 10 to about 50.

A particularly preferred type of filler which may be used is sugars. Sugars include sugar, sugar alcohols, ketoses, saccharides, polysaccharides, oligosaccharides, and the like, as well as celluloses and modified celluloses.

Sugars may also include direct compression and/or nondirect compression sugars. Particularly preferred nondirect compression sugars include, without limitation, dextrose, mannitol, sorbitol, trehalose, lactose, and sucrose. Of course, these sugars generally exist as either a direct compression sugar, i.e., a sugar which has been modified to increase its compressibility and/or flow, or a nondirect compression sugar which does not have sufficient flowability and/or compressibility to allow it to be used in high speed processing and multi-tablet presses without some sort of augmentation such as, without limitation, a glidant to increase flow, granulation to increase flow and/or compressibility and the like. Of course, techniques like granulation can also be used to convert something which initially has sufficient flow and compressibility to be considered a direct compression sugar before processing into a nondirect compression sugar as well. This can be measured by directly compressing tablets made only from a sugar and comparing the flow and compressibility both before and after processing. If flow and/or compressibility are reduced after processing the material is likely to have become a nondirect compression sugar. It will be appreciated however, that whether or not the reduction in properties are sufficient to require augmentation or further processing before the sugar is used in a commercial process will depend on a number of factors including the amount used, the type of processing equipment used, and the overall formulation. Generally, however, some further processing or augmentation is required. While not definitive, sometimes a nondirect compression sugar will have at least about 90% of its particles smaller than about 200 microns, and more preferably 80% smaller than about 150 microns.

The amount of total sugar can range from about 0 to about 90%. More preferably, the amount of sugar will range from about 5% to about 75%, and even more preferably between about 10% and 50%. Other non-carbohydrate diluents and fillers include for example dihydrated or anhydrous calcium carbonate, anhydrous or hydrated calcium sulphate, and calcium lactate trihydrate. When used these are present in an amount of ranging from 0 to about 90%, more preferably from about 5% to about 75% and most preferably from about 10% to about 50% by weight of the dosage form.

Sweeteners for use with the formulations described herein include, e.g., fructose DC; honey DC; maltodextrin; maltose DC; mannitol DC; molasses DC; sorbitol, crystalline; sorbitol, special solution; and sucrose DC. These may be used in conventional amounts.

Artificial sweeteners may also be used and can be anything known to be used as artificial sweeteners. Some artificial sweeteners without limitation include saccharin, aspartame, aspartame and lactose, aspartame dextrose, sucralose, neotame, and acesulfame potassium. Artificial sweeteners may be used in conventional amounts, and preferably in an amount ranging from about 0.1% to about 2%.

Flavoring agents can be anything known to be used as flavoring agents. Flavoring agents may include synthetic flavor oils and flavoring aromatics and/or natural oils, extracts from plants, leaves, flowers, fruits and so forth and combinations thereof. These may include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Also useful as flavoring agents are vanilla, citrus oil, including lemon, orange, banana, grape, lime and grapefruit, and fruit essences, including apple, pear, peach, strawberry, raspberry, cherry, plum, pineapple, apricot and so forth.

Flavoring agents may be used in conventional amounts, and preferably in an amount ranging from about 0.01% to about 3% by weight of the dosage form, and more preferably from about 0.1% to about 2.5% by weight of the dosage form, and most preferably from about 0.25% to about 2% by weight of the dosage form.

Coloring agents can be anything known to be used as a coloring agent. Coloring agents may include titanium dioxide, and dyes suitable for food such as those known as F.D.& C. dyes and natural coloring agents such as grape skin extract, beet red powder, beta-carotene, annatto, carmine, turmeric, paprika, etc. Coloring agents may be used in conventional amounts, and preferably in an amount ranging from about 0.001% to about 1% by weight of the dosage form.

Lubricants can be anything known to be used as a lubricant and include, for example, glycerol palmitostearate, magnesium stearate; stearic acid; calcium stearate; alkaline stearate; talc; and sodium stearyl fumarate. Lubricants may include intrinsic or extrinsic lubricants. Intrinsic lubricants may include magnesium, calcium, zinc salts of stearic acid, hydrogenated and partially hydrogenated vegetable oils, animal fats, polyethylene glycol, polyoxyethylene monostearate, talc, light mineral oils, sodium benzoate, sodium lauryl sulphate, magnesium oxide and the like. Powder lubricants may also be used; nonlimiting examples of powder lubricants include glyceryl behenate. Lubricants may be used in conventional amounts, and preferably in an amount from about 0.1% to about 5% by weight of the dosage form, from about 0.1% to about 3.0% by weight, more preferably from about 0.25% to about 2.5% and most preferably from 0.5% to 2%.

Viscosity modifiers can be anything known to used as a viscosity modifier. Some viscosity modifiers include, without limitation, sodium alginate, hydroxypropyl methylcellulose (HPMC), hydroxyethylcellulose (HEC), sodium carboxymethycellulose (sodium CMC), polyvinylpyrrolidone (PVP), Konjac flour, carrageenan, xanthan gum, other hydrophilic polymers, or mixtures thereof. Viscosity modifiers can be used in conventional amounts and preferably in an amount of about 1% to about 40%, and more preferably in an amount of about 2% to about 20% by weight of the dosage form.

Surfactants can be anything known to be used as surfactants. Some surfactants include, without limitation, various grades of the following commercial products: Arlacel®, Tween®, Capmul®, Centrophase®, Cremophor®, Labrafac®, Labrafil®, Labrasol®, Myverol®, Tagat®, and any non-toxic short and medium chain alcohols. Surfactants can be used in conventional amounts and preferably in an amount of about 0.01% to about 5%, and more preferably in an amount of about 0.1% to about 2% by weight of the dosage form.

Buffers can be anything known to be used as a buffer. Some buffers include any weak acid or weak base or, preferably, any buffer system that is not harmful to the gastrointestinal mucosa. These include, but are not limited to, sodium carbonate, potassium carbonate, disodium hydrogen phosphate, sodium dihydrogen phosphate, and the equivalent potassium salts. Buffers can be used in conventional amounts and preferably in an amount of about 0.1% to about 10%, and more preferably in an amount of about 1% to about 5% by weight of the dosage form.

Disintegrants which may be used include starch, cellulose, modified starch, microcrystalline cellulose, alginic acid, clays, veegum and super disintegrants including, without limitation, cross-linked PVP, croscaramellose salts such as croscaramellose sodium, starch derivatives like sodium starch glycolate, Where such super disintegrants are used, they are traditionally found in an amount of between about 1% and about 20%, more preferably between about 2% and about 10%, and most preferably between about 2% and about 5% by weight of the finished dosage form. In addition to, instead of any portion of, or instead of any super disintegrant, the dosage forms described herein may include at least one effervescent couple or disintegrant. These disintegrants may comprise up to about 20 weight percent and preferably between about 2% and about 10% of the total weight of the dosage form.

Specific disintegrants which may be used include, e.g., crosslinked vinylpyrrolidones (e.g., POLYCLAR AT®), crosslinked carboxymethylcelluloses, crosslinked croscarmelloses (e.g., ADDISOL®), carboxymethylamidons (e.g., AMIGEL®); crospovidone; gellan gum; L-HPC; sodium starch glycolate; and starch DC. These disintegrants, where included, are typically present in an amount between about 0.5 and about 15 percent by weight.

If desired, the dosage form may also contain minor amounts of nontoxic substances such as wetting or emulsifying agents, pH buffering agents and the like, for example, sodium acetate, sorbitan monolaurate, triethanolamine, sodium acetate, triethanolamine oleate, sodium lauryl sulfate, dioctyl sodium sulfosuccinate, polyoxyethylene sorbitan fatty acid esters.

Compression agents/encapsulation aids, where included, are typically present in an amount between 2 and 20% by weight. Nonlimiting examples of compression agents/encapsulation aids include, for example, microcrystalline cellulose (e.g., AVICEL®); PVP of molecular weight 10,000 to 30,000; calcium carbonate; dextrose; fructose; fructose DC; honey DC; lactose anhydrate; lactose monohydrate; lactose and aspartame; lactose and cellulose; lactose and microcrystalline cellulose; maltodextrin; maltose DC; mannitol; microcrystalline cellulose and guar gum; microcrystalline cellulose and lactose; molasses DC; sorbitol, crystalline; starch DC; and, sucrose.

Nonlimiting examples of plasticizers include: dibutyl sebacate; and polyvinylacetate phthalate. Slip/anti-electrostatic agents, where included, are typically present in an amount between 0.1 and 2.0 percent by weight. Non-limiting examples of slip/anti-electrostatic agents include, for example, colloidal silicas (e.g., AEROSIL® 100/200).

In some embodiments, provided are dosage forms (e.g., pills, tablets, caplets, capsules, or powder sachets for oral administration comprising OLC and tenapanor, wherein the dosage forms can be administered without the need to chew or break the product in the mouth before swallowing.

In some embodiments provided are kits (e.g., blister packs) comprising dosage forms comprising OLC and dosage forms comprising tenapanor.

### Methods of Treatment

The inventors have discovered that in addition to potent single agent effects, the novel lanthanum-based phosphate binder OLC has synergistic effects when given in conjunction with tenapanor, an NHE3 blocker that diminishes transcellular phosphate absorption. In a study of rats contemporaneously fed a high phosphorus diet (Example 1), oxylanthanum carbonate, with or without tenapanor, significantly reduced urinary phosphate excretion, and combination OLC+tenapanor synergistically reduced urinary phosphate excretion in rats compared to either treatment alone. Although sample sizes within groups were small, it is noteworthy that the most pronounced synergy (the greatest difference between predicted and observed urinary phosphate excretion) was evident in the lowest OLC dose group (0.75%). Without being bound by theory, one potential explanation is that at higher OLC doses, there may be relatively little phosphate remaining in the intestinal lumen, and as a result, there may be limited incremental benefit of tenapanor. It should also be noted that the OLC+tenapanor combination exhibited four- to seven-fold more synergistic effects compared to the sevelamer+tenapanor combination previously described in the literature (King A J, et al., Am J Physiol Renal Physiol Jan. 1 2021; 320 (1): F133-f144), with the caveats involved in comparing non-contemporaneous studies. Even if combination OLC+tenapanor has similar phosphate lowering effects to high doses of sevelamer and/or other binders, the potency of OLC and the dosing regimen of tenapanor (one small tablet twice daily) could significantly reduce pill burden, below the typical nine to eighteen phosphate binder capsules or tablets ingested by patients with moderate to severe hyperphosphatemia in current practice.

In an embodiment, provided are methods of treatment of a condition characterized by an abnormally elevated level of phosphate in the blood (e.g., selected from the group consisting of hyperphosphatemia, diabetic or non-diabetic chronic kidney disease (CKD), general kidney failure, end stage renal disease (ESRD), and chronic renal insufficiency comprising administering to a patient in need thereof a combination of oxylanthanum carbonate (OLC) and an NHE3 inhibitor (e.g., tenapanor).

In an embodiment, provided are methods of treatment of a condition characterized by an abnormally elevated level of phosphate in the blood (e.g., selected from the group consisting of hyperphosphatemia, diabetic or non-diabetic chronic kidney disease (CKD), general kidney failure, end stage renal disease (ESRD), and chronic renal insufficiency comprising administering to a patient in need thereof a combination of oxylanthanum carbonate (OLC) and tenapanor.

In some embodiments, provided is a method of reducing serum phosphate in a patient with end-stage renal disease (ESRD) in need thereof, the method comprising orally administering to the patient an oxylanthanum carbonate (OLC) and tenapanor.

In some embodiments, provided is a method of reducing serum phosphate in a patient with diabetic or non-diabetic chronic kidney disease (CKD) in need thereof, the method comprising orally administering to the patient an oxylanthanum carbonate (OLC) and tenapanor. In some embodiments, provided is a method of treating hyperphosphatemia in a patient in need thereof, the method comprising orally administering to the patient an oxylanthanum carbonate (OLC) and tenapanor.

In some embodiments, provided is a method of treating hyperphosphatemia in a patient with diabetic or non-diabetic chronic kidney disease (CKD) in need thereof, the method comprising orally administering to the patient an oxylanthanum carbonate (OLC) and tenapanor.

In some embodiments, provided is a method of reducing the incidence and/or severity of side effects associated with administration of phosphorus-lowering treatment in a patient suffering from ESRD in need of phosphorus-lowering treatment comprising orally administering to the patient an oxylanthanum carbonate (OLC) and tenapanor.

In some embodiments, provided is a method of reducing the incidence and/or severity of side effects associated with administration of phosphorus-lowering treatment in a patient suffering from diabetic or non-diabetic CKD in need of phosphorus-lowering treatment comprising orally administering to the patient an oxylanthanum carbonate (OLC) and tenapanor.

In some embodiments, provided is a method of reducing the incidence and/or severity of diarrhea associated with administration of tenapanor in a patient suffering from ESRD in need of tenapanor treatment comprising orally administering to the patient an oxylanthanum carbonate (OLC) and tenapanor.

In some embodiments, provided is a method of reducing the incidence and/or severity of diarrhea associated with administration of tenapanor in a patient suffering from diabetic or non-diabetic CKD in need of tenapanor treatment comprising orally administering to the patient an oxylanthanum carbonate (OLC) and tenapanor.

In some embodiments, provided is a method of reducing the incidence and/or severity of side effects associated with administration of lanthanum-containing phosphorus-reducing treatment in a patient suffering from ESRD in need of treatment with a lanthanum-containing phosphorus-reducing treatment comprising orally administering to the patient an oxylanthanum carbonate (OLC) and tenapanor.

In some embodiments, provided is a method of reducing the incidence and/or severity of side effects associated with administration of lanthanum-containing phosphorus-reducing treatment in a patient suffering from diabetic or non-diabetic CKD in need of treatment with a lanthanum-containing phosphorus-reducing treatment comprising orally administering to the patient an oxylanthanum carbonate (OLC) and tenapanor.

In some embodiments, provided is a method of reducing the incidence and/or severity of side effects associated with administration of phosphorus-lowering treatment in a patient suffering from diabetic or non-diabetic CKD in need of phosphorus-lowering treatment comprising orally administering to the patient an oxylanthanum carbonate (OLC) and tenapanor.

In some embodiments, provided is a method of reducing the incidence and/or severity of side effects associated with administration of phosphorus-lowering treatment in a patient suffering from ESRD in need of phosphorus-lowering treatment comprising orally administering to the patient an oxylanthanum carbonate (OLC) and tenapanor.

In some embodiments, provided is a method of reducing the incidence and/or severity of side effects associated with administration of phosphorus-lowering treatment in a patient suffering from diabetic or non-diabetic CKD in need of phosphorus-lowering treatment comprising orally administering to the patient an oxylanthanum carbonate (OLC) and tenapanor.

In some embodiments, provided is a method of reducing the incidence and/or severity of diarrhea associated with administration of tenapanor in a patient suffering from ESRD in need of tenapanor treatment comprising orally administering to the patient an oxylanthanum carbonate (OLC) and tenapanor.

In some embodiments, provided is a method of reducing the incidence and/or severity of diarrhea associated with administration of tenapanor in a patient suffering from diabetic or non-diabetic CKD in need of tenapanor treatment comprising orally administering to the patient an oxylanthanum carbonate (OLC) and tenapanor.

In some embodiments, provided is a method of reducing the incidence and/or severity of side effects associated with administration of lanthanum-containing phosphorus-reducing treatment in a patient suffering from ESRD in need of treatment with a lanthanum-containing phosphorus-reducing treatment comprising orally administering to the patient an oxylanthanum carbonate (OLC) and tenapanor.

In some embodiments, provided is a method of reducing the incidence and/or severity of side effects associated with administration of lanthanum-containing phosphorus-reducing treatment in a patient suffering from diabetic or non-diabetic CKD in need of treatment with a lanthanum-containing phosphorus-reducing treatment comprising orally administering to the patient an oxylanthanum carbonate (OLC) and tenapanor.

In some embodiments, the CKD is diabetic CKD. In some embodiments, the CKD is non-diabetic CKD.

In some embodiments, the incidence of side effects (e.g., diarrhea) is reduced by at least 20% compared to administration of 30 mg tenapanor BID as described in tenapanor's prescribing information. In some embodiments, the incidence of side effects (e.g., diarrhea) is reduced by at least 30%. In some embodiments, the incidence of side effects (e.g., diarrhea) is reduced by at least 40%.

In some embodiments, tenapanor and the OLC are administered substantially concurrently. In some embodiments, tenapanor and the OLC are administered sequentially. In some embodiments, the OLC and tenapanor are administered on the same dosing schedule. In some embodiments, the OLC and tenapanor are administered on different dosing schedules.

In some embodiments, tenapanor and the OLC are administered substantially concurrently, once daily (QD). In some embodiments, tenapanor and the OLC are administered substantially concurrently, twice daily (BID). In some embodiments, tenapanor and the OLC are administered substantially concurrently, thrice daily (TID). In some embodiments, the tenapanor and OLC are administered substantially concurrently with a meal.

In some embodiments, tenapanor and the OLC are administered as separate dosage forms. In some embodiments, tenapanor and the OLC are administered in a single dosage form.

In some embodiments, tenapanor and the OLC are administered in synergistically effective amounts.

In some embodiments of the methods described herein, administration of the combination of OLC and tenapanor results in similar or lower levels of serum phosphate and lower incidence and/or severity of side effects (e.g., diarrhea) compared to administration of tenapanor in accordance with the XPHOZAH® label. In some embodiments of the methods described herein, administration of the combination of OLC and tenapanor results in similar or lower levels of serum phosphate and lower incidence and/or severity of side effects (e.g., diarrhea) compared to administration of 30 mg BID tenapanor in accordance with the XPHOZAH® label. In some embodiments, the incidence of side effects (e.g., diarrhea) is reduced by at least 20% compared to administration of 30 mg tenapanor BID as described in tenapanor's prescribing information. In some embodiments, the incidence of side effects (e.g., diarrhea) is reduced by at least 30%. In some embodiments, the incidence of side effects (e.g., diarrhea) is reduced by at least 40%.

In some embodiments of the methods described herein, administration of the combination of OLC and tenapanor results in lower serum phosphate levels compared to administration of tenapanor in accordance with the XPHOZAH® label. In some embodiments of the methods described herein, administration of the combination of OLC and tenapanor results in lower serum phosphate levels compared to administration of 30 mg BID tenapanor in accordance with the XPHOZAH® label. In some embodiments, the serum phosphate levels are at least 10% lower. In some embodiments, the serum phosphate levels are at least 20% lower. In some embodiments, the serum phosphate levels are at least 30% lower. In some embodiments, the serum phosphate levels are at least 40% lower. In some embodiments, the serum phosphate levels are at least 50% lower. In some embodiments, the serum phosphate levels are at least 40% lower. In some embodiments, the serum phosphate levels are at least 0.5 mg/dL lower. In some embodiments, the serum phosphate levels are at least 0.75 mg/dL lower. In some embodiments, the serum phosphate levels are at least 1 mg/dL lower. In some embodiments, the serum phosphate levels are at least 1.25 mg/dL lower. In some embodiments, the serum phosphate levels are at least 1.5 mg/dL lower. In some embodiments, the serum phosphate levels are at least 2 mg/dL lower. In some embodiments, the serum phosphate levels are at least 2.5 mg/dL lower. In some embodiments, the serum phosphate levels are at least 3 mg/dL lower. In some embodiments, the serum phosphate levels are at least 3.5 mg/dL lower.

In some embodiments of the methods described herein, administration of the combination of OLC and tenapanor results in lower serum phosphate levels compared to administration of OLC alone. In some embodiments, the serum phosphate levels are at least 10% lower. In some embodiments, the serum phosphate levels are at least 20% lower. In some embodiments, the serum phosphate levels are at least 30% lower. In some embodiments, the serum phosphate levels are at least 50% lower. In some embodiments, the serum phosphate levels are at least 40% lower. In some embodiments, the serum phosphate levels are at least 0.5 mg/dL lower. In some embodiments, the serum phosphate levels are at least 0.75 mg/dL lower. In some embodiments, the serum phosphate levels are at least 1 mg/dL lower. In some embodiments, the serum phosphate levels are at least 1.25 mg/dL lower. In some embodiments, the serum phosphate levels are at least 1.5 mg/dL lower. In some embodiments, the serum phosphate levels are at least 2 mg/dL lower. In some embodiments, the serum phosphate levels are at least 2.5 mg/dL lower. In some embodiments, the serum phosphate levels are at least 3 mg/dL lower. In some embodiments, the serum phosphate levels are at least 3.5 mg/dL lower.

In some embodiments of the methods described herein, administration of the combination of OLC and tenapanor results in lower serum phosphate levels compared to administration of OLC alone at the same OLC dose. In some embodiments, the serum phosphate levels are at least 10% lower. In some embodiments, the serum phosphate levels are at least 20% lower. In some embodiments, the serum phosphate levels are at least 30% lower. In some embodiments, the serum phosphate levels are at least 50% lower. In some embodiments, the serum phosphate levels are at least 40% lower. In some embodiments, the serum phosphate levels are at least 0.5 mg/dL lower. In some embodiments, the serum phosphate levels are at least 0.75 mg/dL lower. In some embodiments, the serum phosphate levels are at least 1 mg/dL lower. In some embodiments, the serum phosphate levels are at least 1.25 mg/dL lower. In some embodiments, the serum phosphate levels are at least 1.5 mg/dL lower. In some embodiments, the serum phosphate levels are at least 2 mg/dL lower. In some embodiments, the serum phosphate levels are at least 2.5 mg/dL lower. In some embodiments, the serum phosphate levels are at least 3 mg/dL lower. In some embodiments, the serum phosphate levels are at least 3.5 mg/dL lower.

In some embodiments of the methods described herein of the methods described herein, the administration of the OLC and tenapanor together result in a urinary phosphorus excretion in the patient that is lower compared to a urinary phosphorus excretion in a patient administered OLC or tenapanor alone. In some embodiments of the methods described herein, the administration of the OLC and tenapanor together result in a urinary phosphorus excretion in the patient after 10 days of daily administration that is lower compared to a urinary phosphorus excretion in a patient administered OLC or tenapanor alone. In some embodiments of the methods described herein, the administration of the OLC and tenapanor together result in a urinary phosphorus excretion in the patient that is at least 10% lower compared to a urinary phosphorus excretion in a patient administered OLC or tenapanor alone. In some embodiments of the methods described herein, the administration of the OLC and tenapanor together result in a urinary phosphorus excretion in the patient that is at least 20% lower compared to a urinary phosphorus excretion in a patient administered OLC or tenapanor alone. In some embodiments of the methods described herein, the administration of the OLC and tenapanor together result in a urinary phosphorus excretion in the patient that is at least 30% lower compared to a urinary phosphorus excretion in a patient administered OLC or tenapanor alone. In some embodiments of the methods described herein, the administration of the OLC and tenapanor together result in a urinary phosphorus excretion in the patient that is at least 40% lower compared to a urinary phosphorus excretion in a patient administered OLC or tenapanor alone. In some embodiments of the methods described herein, the administration of the OLC and tenapanor together result in a urinary phosphorus excretion in the patient that is between 10% and 50% lower compared to a urinary phosphorus excretion in a patient administered OLC or tenapanor alone. In some embodiments of the methods described herein, the administration of the OLC and tenapanor together result in a urinary phosphorus excretion in the patient that is between 10% and 40% lower compared to a urinary phosphorus excretion in a patient administered OLC or tenapanor alone. In some embodiments of the methods described herein, the administration of the OLC and tenapanor together result in a urinary phosphorus excretion in the patient that is between 10% and 30% lower compared to a urinary phosphorus excretion in a patient administered OLC or tenapanor

In some embodiments of the methods described herein, administration of the combination of OLC and tenapanor results in similar or lower serum phosphate levels compared to administration of a dose of single agent OLC that is higher than the OLC dose used in the combination. In some embodiments, the single agent OLC dose is at least 25% higher. In some embodiments, the single agent OLC dose is at least 30% higher. In some embodiments, the single agent OLC dose is at least 40% higher. In some embodiments, the single agent OLC dose is at least 40% higher. In some embodiments, the single agent OLC dose is at least 50% higher. In some embodiments, the single agent OLC dose is at least 75% higher. In some embodiments, the single agent OLC dose is at least 100% higher than the dose used in the combination.

The doses of OLC and tenapanor contemplated for use in the methods described herein are any of the doses described in the “Oxylanthanum carbonate (OLC)” and “NHE3 inhibitors” sections in any combination.

In some embodiments of the methods described herein, the OLC and tenapanor are administered together as a fixed dose dosage form containing any of the doses described herein in any combination.

Throughout the specification, references to methods of treatment (e.g., “a method of treatment,” “a method of treating,” “a method of reducing the incidence and/or severity of side effects,” “a method for reducing the incidence and/or severity of diarrhea,” “a method of reducing serum phosphate,” and the like are meant to also encompass:
a) compounds, compositions and/or combinations of compounds and compositions described herein for use in the respective methods (e.g., “a compound, composition or combination as described herein for use in a method of treatment,” “a compound, composition or combination as described herein for use in a method of treating,” “a compound, composition or combination as described herein for use in a method of reducing the incidence and/or severity of side effects,” “a compound, composition or combination as described herein for use in a method for reducing the incidence and/or severity of diarrhea,” “a compound, composition or combination as described herein for use in a method of reducing serum phosphate”);
b) use of the compounds, compositions and/or combinations of compounds and compositions described herein in the respective methods (e.g., “use of a compound, composition or combination as described herein in a method of treatment,” “use of a compound, composition or combination as described herein in a method of treating,” “use of a compound, composition or combination as described herein in a method of reducing the incidence and/or severity of side effects,” “use of a compound, composition or combination as described herein in a method for reducing the incidence and/or severity of diarrhea,” “use of a compound, composition or combination as described herein in a method of reducing serum phosphate”);
c) use of the compounds, compositions and/or combinations of compounds and compositions described herein in the manufacturing of a medicament for accomplishing the respective methods (e.g., “use of a compound, composition or combination as described herein in the manufacturing of a medicament for treatment,” “use of a compound, composition or combination as described herein in the manufacturing of a medicament for treating,” “use of a compound, composition or combination as described herein in the manufacturing of a medicament for reducing the incidence and/or severity of side effects,” “use of a compound, composition or combination as described herein in the manufacturing of a medicament for reducing the incidence and/or severity of diarrhea,” “use of a compound, composition or combination as described herein in the manufacturing of a medicament for reducing serum phosphate”);
d) Compounds, compositions and/or combinations of compounds and compositions described herein for use in the manufacturing of a medicament for accomplishing the respective methods (e.g., “a compound, composition or combination as described herein for use in the manufacturing of a medicament for treatment,” “a compound, composition or combination as described herein for use in the manufacturing of a medicament for treating,” “a compound, composition or combination as described herein for use in the manufacturing of a medicament for reducing the incidence and/or severity of side effects,” “a compound, composition or combination as described herein for use in the manufacturing of a medicament for reducing the incidence and/or severity of diarrhea,” “a compound, composition or combination as described herein for use in the manufacturing of a medicament for reducing serum phosphate”);

### SELECTED EMBODIMENTS

Embodiment 1. A method of reducing serum phosphate in a patient with end-stage renal disease (ESRD) in need thereof, the method comprising orally administering to the patient an oxylanthanum carbonate (OLC) and tenapanor.

Embodiment 2. A method of reducing serum phosphate in a patient with diabetic or non-diabetic chronic kidney disease (CKD) in need thereof, the method comprising orally administering to the patient an oxylanthanum carbonate (OLC) and tenapanor.

Embodiment 3. A method of treating hyperphosphatemia in a patient in need thereof, the method comprising orally administering to the patient an oxylanthanum carbonate (OLC) and tenapanor.

Embodiment 4. A method of treating hyperphosphatemia in a patient with diabetic or non-diabetic chronic kidney disease (CKD) in need thereof, the method comprising orally administering to the patient an oxylanthanum carbonate (OLC) and tenapanor.

Embodiment 5. A method of reducing the incidence and/or severity of side effects associated with administration of phosphorus-lowering treatment in a patient suffering from ESRD in need of phosphorus-lowering treatment comprising orally administering to the patient an oxylanthanum carbonate (OLC) and tenapanor.

Embodiment 6. A method of reducing the incidence and/or severity of side effects associated with administration of phosphorus-lowering treatment in a patient suffering from diabetic or non-diabetic chronic kidney disease (CKD) in need of phosphorus-lowering treatment comprising orally administering to the patient an oxylanthanum carbonate (OLC) and tenapanor.

Embodiment 7. A method of reducing the incidence and/or severity of diarrhea associated with administration of tenapanor in a patient suffering from ESRD in need of tenapanor treatment comprising orally administering to the patient an oxylanthanum carbonate (OLC) and tenapanor.

Embodiment 8. A method of reducing the incidence and/or severity of diarrhea associated with administration of tenapanor in a patient suffering from diabetic or non-diabetic CKD in need of tenapanor treatment comprising orally administering to the patient an oxylanthanum carbonate (OLC) and tenapanor.

Embodiment 9. A method of reducing the incidence and/or severity of side effects associated with administration of lanthanum-containing phosphorus-reducing treatment in a patient suffering from ESRD in need of treatment with a lanthanum-containing phosphorus-reducing treatment comprising orally administering to the patient an oxylanthanum carbonate (OLC) and tenapanor.

Embodiment 10. A method of reducing the incidence and/or severity of side effects associated with administration of lanthanum-containing phosphorus-reducing treatment in a patient suffering from diabetic or non-diabetic CKD in need of treatment with a lanthanum-containing phosphorus-reducing treatment comprising orally administering to the patient an oxylanthanum carbonate (OLC) and tenapanor.

Embodiment 11. The method of any one of embodiments 1-10, wherein upon administration of the oxylanthanum carbonate (OLC) and tenapanor to the patient, the incidence and/or severity of side effects is lower compared to administration to the patient of tenapanor in accordance with the XPHOZAH® label.

Embodiment 12. The method of any one of embodiments 1-10, wherein upon administration of the oxylanthanum carbonate (OLC) and tenapanor to the patient, the incidence and/or severity of side effects is lower compared to administration to the patient of 30 mg of tenapanor BID.

Embodiment 13. The method of any one of embodiments 1-10, wherein upon administration of the oxylanthanum carbonate (OLC) and the tenapanor to the patient, the incidence and/or severity of diarrhea is lower compared to administration to the patient of tenapanor in accordance with the XPHOZAH® label.

Embodiment 14. The method of any one of embodiments 1-10, wherein upon administration of the oxylanthanum carbonate (OLC) and the tenapanor to the patient, the incidence and/or severity of diarrhea is lower compared to administration to the patient of 30 mg of tenapanor BID.

Embodiment 15. The method of any one of embodiments 1-10, wherein upon administration of the oxylanthanum carbonate (OLC) and the tenapanor to the patient, the incidence and/or severity of side effects is lower compared to administration to the patient of 1000 mg OLC TID.

Embodiment 16. A method of reducing the incidence and/or severity of side effects associated with administration of phosphorus-lowering treatment in a patient suffering from ESRD in need of phosphorus-lowering treatment comprising orally administering to the patient an oxylanthanum carbonate (OLC) and tenapanor.

Embodiment 17. A method of reducing the incidence and/or severity of side effects associated with administration of phosphorus-lowering treatment in a patient suffering from diabetic or non-diabetic CKD in need of phosphorus-lowering treatment comprising orally administering to the patient an oxylanthanum carbonate (OLC) and tenapanor.

Embodiment 18. A method of reducing the incidence and/or severity of diarrhea associated with administration of tenapanor in a patient suffering from ESRD in need of tenapanor treatment comprising orally administering to the patient an oxylanthanum carbonate (OLC) and tenapanor.

Embodiment 19. A method of reducing the incidence and/or severity of diarrhea associated with administration of tenapanor in a patient suffering from diabetic or non-diabetic CKD in need of tenapanor treatment comprising orally administering to the patient an oxylanthanum carbonate (OLC) and tenapanor.

Embodiment 20. A method of reducing the incidence and/or severity of side effects associated with administration of lanthanum-containing phosphorus-reducing treatment in a patient suffering from ESRD in need of treatment with a lanthanum-containing phosphorus-reducing treatment comprising orally administering to the patient an oxylanthanum carbonate (OLC) and tenapanor.

Embodiment 21. A method of reducing the incidence and/or severity of side effects associated with administration of lanthanum-containing phosphorus-reducing treatment in a patient suffering from diabetic or non-diabetic CKD in need of treatment with a lanthanum-containing phosphorus-reducing treatment comprising orally administering to the patient an oxylanthanum carbonate (OLC) and tenapanor.

Embodiment 22. The method of any one of embodiments 1-21, wherein the tenapanor and the OLC are administered in synergistically effective amounts.

Embodiment 23. The method of any one of embodiments 1-22, wherein the OLC is administered at a dose of 1000 mg once daily (QD), twice daily (BID) or three times daily (TID).

Embodiment 24. The method of any one of embodiments 1-22, wherein the OLC is administered at a dose lower than 1000 mg QD, BID or TID.

Embodiment 25. The method of any one of embodiments 1-22, wherein OLC is administered at a dose of 500 mg QD, BID or TID.

Embodiment 26. The method of any one of embodiments 1-22, wherein OLC is administered at a dose lower than 500 mg QD, BID or TID.

Embodiment 27. The method of any one of embodiments 1-22, wherein the OLC is administered at a dose between 100 mg and 1000 mg QD, BID or TID.

Embodiment 28. The method of any one of embodiments 1-22, wherein the OLC is administered at a dose between 100 mg and 750 mg QD, BID or TID.

Embodiment 29. The method of any one of embodiments 1-22, wherein the OLC is administered at a dose between 100 mg and 500 mg QD, BID or TID.

Embodiment 30. The method of any one of embodiments 1-22, wherein the OLC is administered at a dose between 100 mg and 250 mg of OLC QD, BID or TID.

Embodiment 31. The method of any one of embodiments 1-22, wherein the OLC is administered at a dose between 250 mg and 1000 mg QD, BID or TID.

Embodiment 32. The method of any one of embodiments 1-22, wherein the OLC is administered at a dose between 250 mg and 750 mg QD, BID or TID.

Embodiment 33. The method of any one of embodiments 1-22, wherein the OLC is administered at a dose between 250 mg and 500 mg QD, BID or TID.

Embodiment 34. The method of any one of embodiments 1-22, wherein the OLC is administered at a dose between 500 mg and 1000 mg QD, BID or TID.

Embodiment 35. The method of any one of embodiments 1-22, wherein the OLC is administered at a dose between 500 mg and 750 mg QD, BID or TID.

Embodiment 36. The method of any one of embodiments 1-22, wherein the OLC is administered at a dose between 500 mg and 750 mg QD, BID or TID.

Embodiment 37. The method of any one of embodiments 1-22, wherein the OLC is administered at a dose of 100 mg, 125 mg, 150 mg, 250 mg, 500 mg, 750 mg, or 1000 mg QD, BID or TID.

Embodiment 38. The method of any one of embodiments 1-22, wherein the OLC is administered at a dose of 100 mg, 125 mg, 150 mg, 250 mg, 500 mg, or 750 mg QD, BID or TID.

Embodiment 39. The method of any one of embodiments 1-22, wherein the OLC is administered at a dose of 100 mg, 125 mg, 150 mg, 250 mg, or 500 mg QD, BID or TID.

Embodiment 40. The method of any one of embodiments 1-22, wherein the OLC is administered at a dose of 250 mg, 500 mg, 750 mg, or 1000 mg QD, BID or TID.

Embodiment 41. The method of any one of embodiments 1-22, wherein the OLC is administered at a dose of 250 mg, 500 mg, or 750 mg QD, BID or TID.

Embodiment 42. The method of any one of embodiments 1-22, wherein the OLC is administered at a dose of 250 mg or 500 mg QD, BID or TID.

Embodiment 43. The method of any one of embodiments 1-42, wherein the OLC is administered QD.

Embodiment 44. The method of any one of embodiments 1-42, wherein the OLC is administered BID or TID.

Embodiment 45. The method of any one of embodiments 1-42, wherein the OLC is administered BID.

Embodiment 46. The method of any one of embodiments 1-42, wherein the OLC is administered TID.

Embodiment 47. The method of any one of embodiments 1-46, wherein the tenapanor is administered at a dose of 30 mg QD, BID or TID.

Embodiment 48. The method of any one of embodiments 1-46, wherein the tenapanor is administered at a dose lower than 30 mg QD, BID or TID.

Embodiment 49. The method of any one of embodiments 1-46, wherein the tenapanor is administered at a dose between 0.1 mg and 25 mg QD, BID or TID.

Embodiment 50. The method of any one of embodiments 1-46, wherein the tenapanor is administered at a dose between 0.1 mg and 20 mg QD, BID or TID.

Embodiment 51. The method of any one of embodiments 1-46, wherein the tenapanor is administered at a dose between 0.1 mg and 15 mg QD, BID or TID.

Embodiment 52. The method of any one of embodiments 1-46, wherein the tenapanor is administered at a dose between 0.1 mg and 10 mg QD, BID or TID.

Embodiment 53. The method of any one of embodiments 1-46, wherein the tenapanor is administered at a dose between 0.1 mg and 5 mg QD, BID or TID.

Embodiment 54. The method of any one of embodiments 1-46, wherein the tenapanor is administered at a dose between 0.1 mg and 3 mg QD, BID or TID.

Embodiment 55. The method of any one of embodiments 1-46, wherein the tenapanor is administered at a dose between 1 mg and 25 mg QD, BID or TID.

Embodiment 56. The method of any one of embodiments 1-46, wherein the tenapanor is administered at a dose between 1 mg and 20 mg QD, BID or TID.

Embodiment 57. The method of any one of embodiments 1-46, wherein the tenapanor is administered at a dose between 1 mg and 15 mg QD, BID or TID.

Embodiment 58. The method of any one of embodiments 1-46, wherein the tenapanor is administered at a dose between 1 mg and 10 mg QD, BID or TID.

Embodiment 59. The method of any one of embodiments 1-46, wherein the tenapanor is administered at a dose between 1 mg and 5 mg QD, BID or TID.

Embodiment 60. The method of any one of embodiments 1-46, wherein the tenapanor is administered at a dose between 3 mg and 25 mg QD, BID or TID.

Embodiment 61. The method of any one of embodiments 1-46, wherein the tenapanor is administered at a dose between 3 mg and 20 mg QD, BID or TID.

Embodiment 62. The method of any one of embodiments 1-46, wherein the tenapanor is administered at a dose between 3 mg and 15 mg QD, BID or TID.

Embodiment 63. The method of any one of embodiments 1-46, wherein the tenapanor is administered at a dose between 3 mg and 10 mg QD, BID or TID.

Embodiment 64. The method of any one of embodiments 1-46, wherein the tenapanor is administered at a dose between 5 mg and 25 mg QD, BID or TID.

Embodiment 65. The method of any one of embodiments 1-46, wherein the tenapanor is administered at a dose between 5 mg and 20 mg QD, BID or TID.

Embodiment 66. The method of any one of embodiments 1-46, wherein the tenapanor is administered at a dose between 5 mg and 15 mg QD, BID or TID.

Embodiment 67. The method of any one of embodiments 1-46, wherein the tenapanor is administered at a dose between 5 mg and 10 mg QD, BID or TID.

Embodiment 68. The method of any one of embodiments 1-46, wherein the tenapanor is administered at a dose of 0.1 mg, 0.3 mg, 0.5 mg, 1 mg, 2 mg, 3 mg, 4 mg, 5 mg, 10 mg, 15 mg, or 20 mg QD, BID or TID.

Embodiment 69. The method of any one of embodiments 1-46, wherein the tenapanor is administered at a dose of 0.1 mg, 0.3 mg, 0.5 mg, 1 mg, 2 mg, 3 mg, 4 mg, 5 mg, or 10 mg QD, BID or TID.

Embodiment 70. The method of any one of embodiments 1-46, wherein the tenapanor is administered at a dose of 0.1 mg, 0.3 mg, 0.5 mg, 1 mg, 2 mg, 3 mg, 4 mg, or 5 mg QD, BID or TID.

Embodiment 71. The method of any one of embodiments 1-46, wherein the tenapanor is administered at a dose of 0.3 mg, 0.5 mg, 1 mg, 2 mg, 3 mg, 4 mg, 5 mg, 10 mg, 15 mg, or 20 mg QD, BID or TID.

Embodiment 72. The method of any one of embodiments 1-46, wherein the tenapanor is administered at a dose of 0.3 mg, 0.5 mg, 1 mg, 2 mg, 3 mg, 4 mg, 5 mg, or 10 mg QD, BID or TID.

Embodiment 73. The method of any one of embodiments 1-46, wherein the tenapanor is administered at a dose of 0.5 mg, 1 mg, 2 mg, 3 mg, 4 mg, or 5 mg QD, BID or TID.

Embodiment 74. The method of any one of embodiments 1-46, wherein the tenapanor is administered at a dose of 0.5 mg, 1 mg, 2 mg, 3 mg, 4 mg, 5 mg, 10 mg, 15 mg, or 20 mg QD, BID or TID.

Embodiment 75. The method of any one of embodiments 1-46, wherein the tenapanor is administered at a dose of 0.5 mg, 1 mg, 2 mg, 3 mg, 4 mg, 5 mg, or 10 mg QD, BID or TID.

Embodiment 76. The method of any one of embodiments 1-46, wherein the tenapanor is administered at a dose of 0.5 mg, 1 mg, 2 mg, 3 mg, 4 mg, or 5 mg QD, BID or TID.

Embodiment 77. The method of any one of embodiments 1-46, wherein the tenapanor is administered at a dose of 1 mg, 2 mg, 3 mg, 4 mg, 5 mg, 10 mg, 15 mg, or 20 mg QD, BID or TID.

Embodiment 78. The method of any one of embodiments 1-46, wherein the tenapanor is administered at a dose of 1 mg, 2 mg, 3 mg, 4 mg, 5 mg, or 10 mg QD, BID or TID.

Embodiment 79. The method of any one of embodiments 1-46, wherein the tenapanor is administered at a dose of 1 mg, 2 mg, 3 mg, 4 mg, or 5 mg QD, BID or TID.

Embodiment 80. The method of any one of embodiments 1-46, wherein the tenapanor is administered at a dose of 3 mg, 5 mg, 10 mg, 15 mg, or 20 mg QD, BID or TID.

Embodiment 81. The method of any one of embodiments 1-46, wherein the tenapanor is administered at a dose of 3 mg, 5 mg, or 10 mg QD, BID or TID.

Embodiment 82. The method of any one of embodiments 1-46, wherein the tenapanor is administered at a dose of 3 mg or 5 mg QD, BID or TID.

Embodiment 83. The method of any one of embodiments 1-82, wherein the tenapanor is administered QD.

Embodiment 84. The method of any one of embodiments 1-82, wherein the tenapanor is administered BID.

Embodiment 85. The method of any one of embodiments 1-82, wherein the tenapanor is administered TID.

Embodiment 86. The method of any one of embodiments 1-85, wherein the tenapanor is administered at a dose 3 mg or 5 mg QD, BID or TID.

Embodiment 87. The method of any one of embodiments 1-86, wherein the OLC and tenapanor are administered substantially concurrently to the patient.

Embodiment 88. The method of any one of embodiments 1-86, wherein the OLC and tenapanor are administered sequentially to the patient.

Embodiment 89. The method of any one of embodiments 1-86, wherein the OLC and tenapanor are administered on the same dosing schedule.

Embodiment 90. The method of any one of embodiments 1-86, wherein the OLC and tenapanor are administered on different dosing schedules.

Embodiment 91. The method of any one of embodiments 1-86, wherein the method comprises administering the OLC and tenapanor at the same time, twice daily (BID).

Embodiment 92. The method of any one of embodiments 1-86, wherein the method comprises administering the OLC and tenapanor at the same time, thrice daily (TID).

Embodiment 93. The method of any one of embodiments 1-92, wherein the method comprises administering the OLC and tenapanor by swallowing an intact dosage form containing the tenapanor and an intact dosage form containing the OLC.

Embodiment 94. The method of any one of embodiments 1-86, wherein the method comprises administering the OLC and tenapanor by swallowing an intact dosage form containing the tenapanor and the OLC.

Embodiment 95. The method of any one of embodiments 1-94, wherein the administration of the OLC and tenapanor together result in a serum phosphorus level in the patient that is lower compared to a serum phosphate level in a patient administered OLC or tenapanor alone.

Embodiment 96. The method of any one of embodiments 1-94, wherein the administration of the OLC and tenapanor together result in a serum phosphate level in the patient after 10 days of daily administration that is lower compared to a serum phosphate level in a patient administered OLC or tenapanor alone.

Embodiment 97. The method of embodiment 95 or 96 wherein the administration of the OLC and tenapanor together result in a serum phosphate level in the patient that is at least 10% lower compared to a serum phosphate level in a patient administered OLC or tenapanor alone.

Embodiment 98. The method of embodiment 95 or 96 wherein the administration of the OLC and tenapanor together result in a serum phosphate level in the patient that is at least 20% lower compared to a serum phosphate level in a patient administered OLC or tenapanor alone.

Embodiment 99. The method of embodiment 95 or 96 wherein the administration of the OLC and tenapanor together result in a serum phosphate level in the patient that is at least 30% lower compared to a serum phosphate level in a patient administered OLC or tenapanor alone.

Embodiment 100. The method of embodiment 95 or 967 wherein the administration of the OLC and tenapanor together result in a serum phosphate level in the patient that is at least 40% lower compared to a serum phosphate level in a patient administered OLC or tenapanor alone.

Embodiment 101. The method of embodiment 95 or 96 wherein the administration of the OLC and tenapanor together result in a serum phosphate level in the patient that is between 10% and 50% lower compared to a serum phosphate level in a patient administered OLC or tenapanor alone.

Embodiment 102. The method of embodiment 95 or 96 wherein the administration of the OLC and tenapanor together result in a serum phosphate level in the patient that is between 10% and 40% lower compared to a serum phosphate level in a patient administered OLC or tenapanor alone.

Embodiment 103. The method of embodiment 95 or 96 wherein the administration of the OLC and tenapanor together result in a serum phosphate level in the patient that is between 10% and 30% lower compared to a serum phosphate level in a patient administered OLC or tenapanor.

Embodiment 104. A dosage form for oral administration comprising an effective amount of an oxylanthanum carbonate (OLC) and tenapanor.

Embodiment 105. The dosage form of embodiment 104, wherein the dosage form is a swallowable tablet or caplet.

Embodiment 106. The dosage form of embodiment 104 or 105, wherein the amount of OLC and the amount of tenapanor present in the dosage form are synergistically effective.

Embodiment 107. The dosage form of embodiment 104 or 105, wherein the amount of at least one of the OLC and tenapanor present in the dosage form are lower than the amount approved for use as single agent or, in the case of tenapanor approved for use in combination with other phosphate binders effective.

Embodiment 108. The dosage form of any one of embodiments 104-107, wherein the dosage form comprises OLC at a dose of 1000 mg.

Embodiment 109. The dosage form of any one of embodiments 104-107, wherein the dosage form comprises OLC at a dose lower than 1000 mg.

Embodiment 110. The dosage form of any one of embodiments 104-107, wherein the dosage form comprises OLC at a dose of 500 mg.

Embodiment 111. The dosage form of any one of embodiments 104-107, wherein the dosage form comprises OLC at a dose lower than 500 mg.

Embodiment 112. The dosage form of any one of embodiments 104-107, wherein the dosage form comprises OLC at a dose between 100 mg and 1000 mg.

Embodiment 113. The dosage form of any one of embodiments 104-107, wherein the dosage form comprises OLC at a dose between 100 mg and 750 mg.

Embodiment 114. The dosage form of any one of embodiments 104-107, wherein the dosage form comprises OLC at a dose between 100 mg and 500 mg.

Embodiment 115. The dosage form of any one of embodiments 104-107, wherein the dosage form comprises OLC at a dose between 100 mg and 250 mg of OLC.

Embodiment 116. The dosage form of any one of embodiments 104-107, wherein the dosage form comprises OLC at a dose between 250 mg and 1000 mg.

Embodiment 117. The dosage form of any one of embodiments 104-107, wherein the dosage form comprises OLC at a dose between 250 mg and 750 mg.

Embodiment 118. The dosage form of any one of embodiments 104-107, wherein the dosage form comprises OLC at a dose between 250 mg and 500 mg.

Embodiment 119. The dosage form of any one of embodiments 104-107, wherein the dosage form comprises OLC at a dose between 500 mg and 1000 mg.

Embodiment 120. The dosage form of any one of embodiments 104-107, wherein the dosage form comprises OLC at a dose between 500 mg and 750 mg.

Embodiment 121. The dosage form of any one of embodiments 104-107, wherein the dosage form comprises OLC at a dose between 500 mg and 750 mg.

Embodiment 122. The dosage form of any one of embodiments 104-107, wherein the dosage form comprises OLC at a dose of 100 mg, 125 mg, 150 mg, 250 mg, 500 mg, 750 mg, or 1000 mg.

Embodiment 123. The dosage form of any one of embodiments 104-107, wherein the dosage form comprises OLC at a dose of 100 mg, 125 mg, 150 mg, 250 mg, 500 mg, or 750 mg.

Embodiment 124. The dosage form of any one of embodiments 104-107, wherein the dosage form comprises OLC at a dose of 100 mg, 125 mg, 150 mg, 250 mg, or 500 mg.

Embodiment 125. The dosage form of any one of embodiments 104-107, wherein the dosage form comprises OLC at a dose of 250 mg, 500 mg, 750 mg, or 1000 mg.

Embodiment 126. The dosage form of any one of embodiments 104-107, wherein the dosage form comprises OLC at a dose of 250 mg, 500 mg, or 750 mg.

Embodiment 127. The dosage form of any one of embodiments 104-107, wherein the dosage form comprises OLC at a dose of 250 mg or 500 mg.

Embodiment 128. The dosage form of any one of embodiments 104-127, wherein the dosage form comprises tenapanor at a dose lower than 30 mg.

Embodiment 129. The dosage form of any one of embodiments 104-127, wherein the dosage form comprises tenapanor at a dose between 0.1 mg and 25 mg.

Embodiment 130. The dosage form of any one of embodiments 104-127, wherein the dosage form comprises tenapanor at a dose between 0.1 mg and 20 mg.

Embodiment 131. The dosage form of any one of embodiments 104-127, wherein the dosage form comprises tenapanor at a dose between 0.1 mg and 15 mg.

Embodiment 132. The dosage form of any one of embodiments 104-127, wherein the dosage form comprises tenapanor at a dose between 0.1 mg and 10 mg.

Embodiment 133. The dosage form of any one of embodiments 104-127, wherein the dosage form comprises tenapanor at a dose between 0.1 mg and 5 mg.

Embodiment 134. The dosage form of any one of embodiments 104-127, wherein the dosage form comprises tenapanor at a dose between 0.1 mg and 3 mg.

Embodiment 135. The dosage form of any one of embodiments 104-127, wherein the dosage form comprises tenapanor at a dose between 1 mg and 25 mg.

Embodiment 136. The dosage form of any one of embodiments 104-127, wherein the dosage form comprises tenapanor at a dose between 1 mg and 20 mg.

Embodiment 137. The dosage form of any one of embodiments 104-127, wherein the dosage form comprises tenapanor at a dose between 1 mg and 15 mg.

Embodiment 138. The dosage form of any one of embodiments 104-127, wherein the dosage form comprises tenapanor at a dose between 1 mg and 10 mg.

Embodiment 139. The dosage form of any one of embodiments 104-127, wherein the dosage form comprises tenapanor at a dose between 1 mg and 5 mg.

Embodiment 140. The dosage form of any one of embodiments 104-127, wherein the dosage form comprises tenapanor at a dose between 3 mg and 25 mg.

Embodiment 141. The dosage form of any one of embodiments 104-127, wherein the dosage form comprises tenapanor at a dose between 3 mg and 20 mg.

Embodiment 142. The dosage form of any one of embodiments 104-127, wherein the dosage form comprises tenapanor at a dose between 3 mg and 15 mg.

Embodiment 143. The dosage form of any one of embodiments 104-127, wherein the dosage form comprises tenapanor at a dose between 3 mg and 10 mg.

Embodiment 144. The dosage form of any one of embodiments 104-127, wherein the dosage form comprises tenapanor at a dose between 5 mg and 25 mg.

Embodiment 145. The dosage form of any one of embodiments 104-127, wherein the dosage form comprises tenapanor at a dose between 5 mg and 20 mg.

Embodiment 146. The dosage form of any one of embodiments 104-127, wherein the dosage form comprises tenapanor at a dose between 5 mg and 15 mg.

Embodiment 147. The dosage form of any one of embodiments 104-127, wherein the dosage form comprises tenapanor at a dose between 5 mg and 10 mg.

Embodiment 148. The dosage form of any one of embodiments 104-127, wherein the dosage form comprises tenapanor at a dose of 0.1 mg, 0.3 mg, 0.5 mg, 1 mg, 2 mg, 3 mg, 4 mg, 5 mg, 10 mg, 15 mg, or 20 mg.

Embodiment 149. The dosage form of any one of embodiments 104-127, wherein the dosage form comprises tenapanor at a dose of 0.1 mg, 0.3 mg, 0.5 mg, 1 mg, 2 mg, 3 mg, 4 mg, 5 mg, or 10 mg.

Embodiment 150. The dosage form of any one of embodiments 104-127, wherein the dosage form comprises tenapanor at a dose of 0.1 mg, 0.3 mg, 0.5 mg, 1 mg, 2 mg, 3 mg, 4 mg, or 5 mg.

Embodiment 151. The dosage form of any one of embodiments 104-127, wherein the dosage form comprises tenapanor at a dose of 0.3 mg, 0.5 mg, 1 mg, 2 mg, 3 mg, 4 mg, 5 mg, 10 mg, 15 mg, or 20 mg.

Embodiment 152. The dosage form of any one of embodiments 104-127, wherein the dosage form comprises tenapanor at a dose of 0.3 mg, 0.5 mg, 1 mg, 2 mg, 3 mg, 4 mg, 5 mg, or 10 mg.

Embodiment 153. The dosage form of any one of embodiments 104-127, wherein the dosage form comprises tenapanor at a dose of 0.5 mg, 1 mg, 2 mg, 3 mg, 4 mg, or 5 mg.

Embodiment 154. The dosage form of any one of embodiments 104-127, wherein the dosage form comprises tenapanor at a dose of 0.5 mg, 1 mg, 2 mg, 3 mg, 4 mg, 5 mg, 10 mg, 15 mg, or 20 mg.

Embodiment 155. The dosage form of any one of embodiments 104-127, wherein the dosage form comprises tenapanor at a dose of 0.5 mg, 1 mg, 2 mg, 3 mg, 4 mg, 5 mg, or 10 mg.

Embodiment 156. The dosage form of any one of embodiments 104-127, wherein the dosage form comprises tenapanor at a dose of 0.5 mg, 1 mg, 2 mg, 3 mg, 4 mg, or 5 mg.

Embodiment 157. The dosage form of any one of embodiments 104-127, wherein the dosage form comprises tenapanor at a dose of 1 mg, 2 mg, 3 mg, 4 mg, 5 mg, 10 mg, 15 mg, or 20 mg.

Embodiment 158. The dosage form of any one of embodiments 104-127, wherein the dosage form comprises tenapanor at a dose of 1 mg, 2 mg, 3 mg, 4 mg, 5 mg, or 10 mg.

Embodiment 159. The dosage form of any one of embodiments 104-127, wherein the dosage form comprises tenapanor at a dose of 1 mg, 2 mg, 3 mg, 4 mg, or 5 mg.

Embodiment 160. The dosage form of any one of embodiments 104-127, wherein the dosage form comprises tenapanor at a dose of 3 mg, 5 mg, 10 mg, 15 mg, or 20 mg.

Embodiment 161. The dosage form of any one of embodiments 104-127, wherein the dosage form comprises tenapanor at a dose of 3 mg, 5 mg, or 10 mg.

Embodiment 162. The dosage form of any one of embodiments 104-127, wherein the dosage form comprises tenapanor at a dose of 3 mg or 5 mg.

Embodiment 163. A method of reducing serum phosphate in a patient with end-stage renal disease (ESRD) in need thereof, the method comprising orally administering to the patient the dosage form of any one of embodiments 104-162.

Embodiment 164. A method of reducing serum phosphate in a patient with diabetic or non-diabetic chronic kidney disease (CKD) in need thereof, the method comprising orally administering to the patient the dosage form of any one of embodiments 104-162.

Embodiment 165. A method of treating hyperphosphatemia in a patient in need thereof, the method comprising orally administering to the patient the dosage form of any one of embodiments 104-162.

Embodiment 166. A method of treating hyperphosphatemia in a patient with diabetic or non-diabetic chronic kidney disease (CKD) in need thereof, the method comprising orally administering to the patient the dosage form of any one of embodiments 104-162.

Embodiment 167. A method of reducing the incidence and/or severity of side effects associated with administration of phosphorus-lowering treatment in a patient suffering from ESRD in need of phosphorus-lowering treatment comprising orally administering to the patient the dosage form of any one of embodiments 104-162.

Embodiment 168. A method of reducing the incidence and/or severity of side effects associated with administration of phosphorus-lowering treatment in a patient suffering from diabetic or non-diabetic chronic kidney disease (CKD) in need of phosphorus-lowering treatment comprising orally administering to the patient the dosage form of any one of embodiments 104-162.

Embodiment 169. A method of reducing the incidence and/or severity of diarrhea associated with administration of tenapanor in a patient suffering from ESRD in need of tenapanor treatment comprising orally administering to the patient the dosage form of any one of embodiments 104-162.

Embodiment 170. A method of reducing the incidence and/or severity of diarrhea associated with administration of tenapanor in a patient suffering from diabetic or non-diabetic CKD in need of tenapanor treatment comprising orally administering to the patient the dosage form of any one of embodiments 104-162.

Embodiment 171. A method of reducing the incidence and/or severity of side effects associated with administration of lanthanum-containing phosphorus-reducing treatment in a patient suffering from ESRD in need of treatment with a lanthanum-containing phosphorus-reducing treatment comprising orally administering to the patient the dosage form of any one of embodiments 104-162.

Embodiment 172. A method of reducing the incidence and/or severity of side effects associated with administration of lanthanum-containing phosphorus-reducing treatment in a patient suffering from diabetic or non-diabetic CKD in need of treatment with a lanthanum-containing phosphorus-reducing treatment comprising orally administering to the patient the dosage form of any one of embodiments 104-162.

Embodiment 173. The method of any one of embodiments 163-172, wherein upon administration of the dosage form to the patient, the incidence and/or severity of side effects is lower compared to administration to the patient of tenapanor in accordance with the XPHOZAH® label.

Embodiment 174. The method of any one of embodiments 163-172, wherein upon administration of dosage form to the patient, the incidence and/or severity of side effects is lower compared to administration to the patient of 30 mg of tenapanor BID.

Embodiment 175. The method of any one of embodiments 163-172, wherein upon administration of the dosage form to the patient, the incidence and/or severity of diarrhea is lower compared to administration to the patient of tenapanor in accordance with the XPHOZAH® label.

Embodiment 176. The method of any one of embodiments 163-172, wherein upon administration of the dosage form to the patient, the incidence and/or severity of diarrhea is lower compared to administration to the patient of 30 mg of tenapanor BID.

Embodiment 177. The method of any one of embodiments 163-172, wherein upon administration of the dosage form to the patient, the incidence and/or severity of side effects is lower compared to administration to the patient of 1000 mg OLC TID.

Embodiment 178. A method of reducing the incidence and/or severity of side effects associated with administration of phosphorus-lowering treatment in a patient suffering from ESRD in need of phosphorus-lowering treatment comprising orally administering to the patient the dosage form of any one of embodiments 104-162.

Embodiment 179. A method of reducing the incidence and/or severity of side effects associated with administration of phosphorus-lowering treatment in a patient suffering from diabetic or non-diabetic CKD in need of phosphorus-lowering treatment comprising orally administering to the patient the dosage form of any one of embodiments 104-162.

Embodiment 180. A method of reducing the incidence and/or severity of diarrhea associated with administration of tenapanor in a patient suffering from ESRD in need of tenapanor treatment comprising orally administering to the patient the dosage form of any one of embodiments 104-162.

Embodiment 181. A method of reducing the incidence and/or severity of diarrhea associated with administration of tenapanor in a patient suffering from diabetic or non-diabetic CKD in need of tenapanor treatment comprising orally administering to the patient the dosage form of any one of embodiments 104-162.

Embodiment 182. A method of reducing the incidence and/or severity of side effects associated with administration of lanthanum-containing phosphorus-reducing treatment in a patient suffering from ESRD in need of treatment with a lanthanum-containing phosphorus-reducing treatment comprising orally administering to the patient the dosage form of any one of embodiments 104-162.

Embodiment 183. A method of reducing the incidence and/or severity of side effects associated with administration of lanthanum-containing phosphorus-reducing treatment in a patient suffering from diabetic or non-diabetic CKD in need of treatment with a lanthanum-containing phosphorus-reducing treatment comprising orally administering to the patient the dosage form of any one of embodiments 104-162.

Embodiment 184. The method of any one of embodiments 163-183, wherein the method comprises administering the dosage form once daily (QD).

Embodiment 185. The method of any one of embodiments 163-183, wherein the method comprises administering the dosage form twice daily (BID).

Embodiment 186. The method of any one of embodiments 163-183, wherein the method comprises administering the dosage form thrice daily (TID).

### EXAMPLES

### Example 1. Effect of OLC and Tenapanor Combinations on Phosphorus Excretion in Sprague Dawley Rats

Sixty-four male Sprague Dawley rats of 9 weeks old (11 weeks by the end of the study) were fed standard chow one week prior to study start. On study day-1, animals were randomized into 8 study groups (n=8): 1) vehicle, 2) tenapanor 0.15 mg/kg, 3) Vehicle+OLC 0.75%, 4) Vehicle+OLC 1.5%, 5) Vehicle+OLC 3%, 6) tenapanor 0.15 mg/kg+OLC 0.75%, 7) tenapanor 0.15 mg/kg+OLC 1.5%, and 8) tenapanor 0.15 mg/kg+OLC 3%. Vehicle and tenapanor were dosed PO twice/day whereas OLC was incorporated into the diets. On study Day 1, all animals were switched to high phosphorus diets consisting of chow spiked with 0.4% inorganic phosphorus [(1:1 sodium:potassium salt-wt./wt.), 1.1% total phosphorus content] for the rest of the study. Body weight and food intake were measured daily from study Day-3 until termination. 24-hour urine samples were collected using metabolic cages on day 9, 10, and 11 for urinary P measurements.

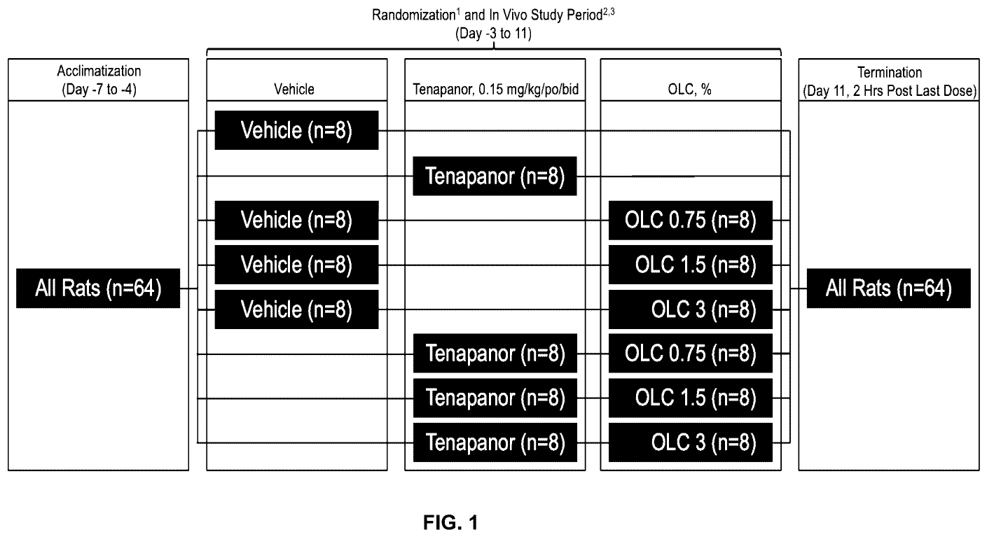

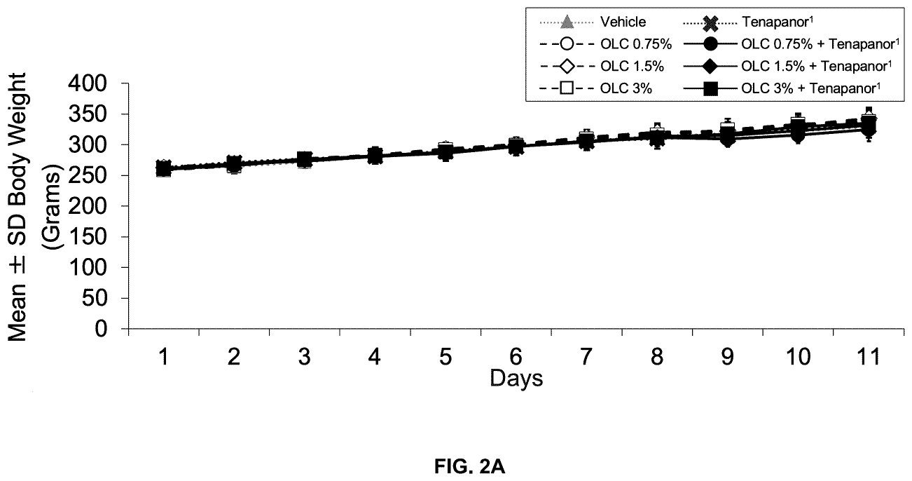

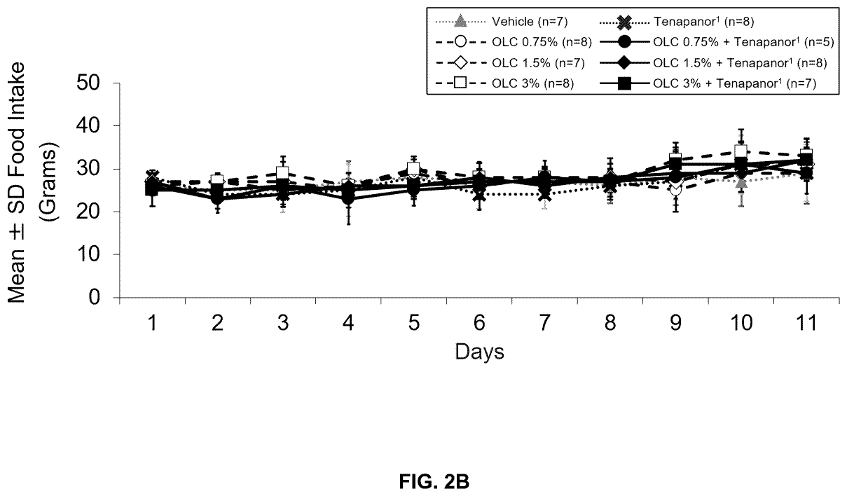

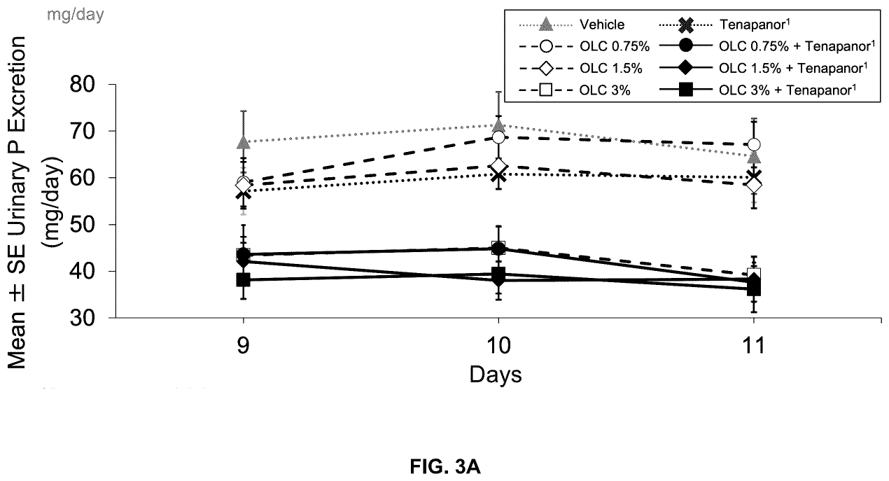

A schematic of the study design is shown in FIG. 1. The data from the experiment is shown in FIG. 2-FIG. 5.

The mean body weight (FIG. 2A) and food intake (FIG. 2B) were measured from Day 1 to 11. Body weight (FIG. 2A) and food intake (FIG. 2B) were not significantly affected by OLC, tenapanor, or combination treatment compared with vehicle.

Mean urinary phosphate excretion for each animal from Days 9 to 11 were averaged by treatment group. While we examined effects separately in each OLC dose group, our primary analyses compared pooled results in the three OLC dose groups combined. We compared pooled results using one-way ANOVA followed by Games-Howell test, and individual groups with vehicle using one-way ANOVA followed by Dunnett's multiple comparison test. A threshold of p<0.05 was used to determine significant differences between groups. Statistical Package for the Social Sciences (SPSS) was used for all statistical analyses.

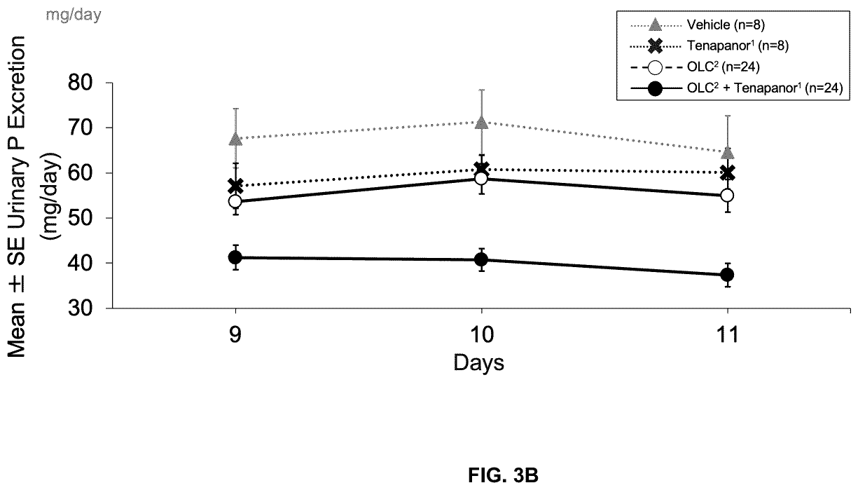

The mean urinary phosphate excretion for each of days 9, 10 and 11 between all 8 study groups is shown in FIG. 3A and pooled in FIG. 3B. OLC dose dependently decreased urinary phosphate excretion. When used together, OLC+tenapanor decreased urinary phosphate excretion such that combination reductions were larger compared to vehicle, OLC only, and tenapanor only.

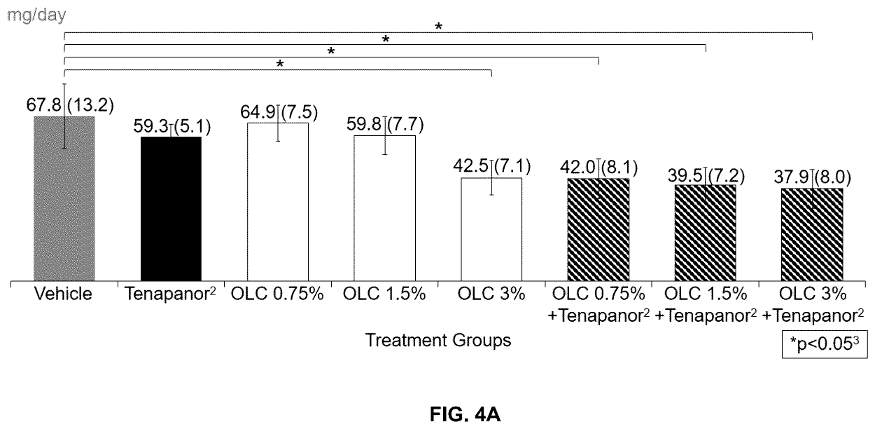

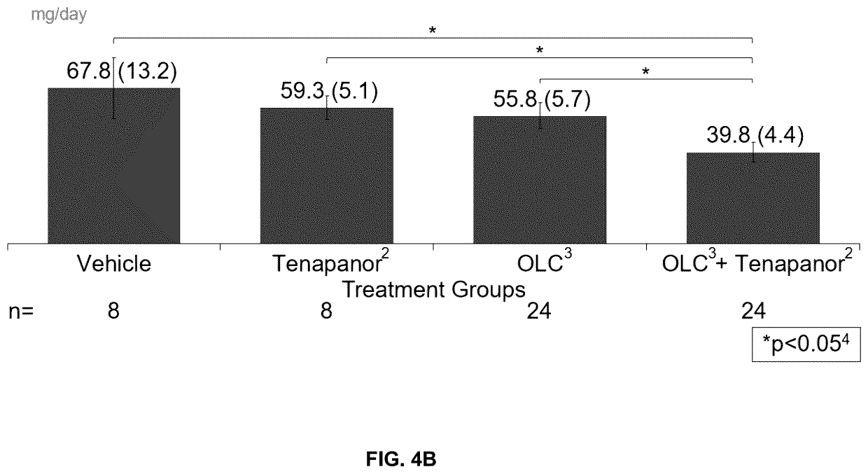

Mean urinary phosphate excretion from Day 9 to 11 by each dose group is shown in FIG. 4A with results pooled across the 3 OLC doses shown in FIG. 4B. In the tenapanor 0.15 mg/kg group, mean urinary phosphate excretion from Days 9 to 11 was 8.5 mg/day lower compared to the vehicle group. In the OLC alone groups, mean urinary phosphate excretion (pooled across the 0.75, 1.5, and 3% OLC dose groups) was 12 mg/day lower compared to the vehicle group. In the OLC+tenapanor groups (pooled across the 0.75, 1.5, and 3% OLC dose groups), urinary phosphate excretion was 28 mg/day lower compared to the vehicle group (p=0.016). Post-hoc Games-Howell test results comparing mean urinary phosphate excretion are shown in Table 1.

TABLE 1

| | | | | | 95% Confidence | |
|---|---|---|---|---|---|---|
| | | Mean | | | Interval | |
| Treatment | Treatment | Difference | | | Lower | Upper |
| Group (A) | Group (B) | (A − B) | Std. Error | Sig. | Bound | Bound |
| Vehicle | Tenapanor³ | 8.510 | 7.215 | 0.653 | −13.997 | 31.017 |
| | OLC² | 12.093 | 7.327 | 0.397 | −10.441 | 34.626 |
| | OLC² + | 28.053 | 7.090 | 0.016 | 5.698 | 50.409 |
| | Tenapanor³ | | | | | |
| Tenapanor³ | Vehicle | −8.510 | 7.215 | 0.653 | −31.017 | 13.997 |
| | OLC² | 3.583 | 3.884 | 0.793 | −7.134 | 14.299 |
| | OLC² + | 19.543 | 3.417 | <0.001 | 9.892 | 29.195 |
| | Tenapanor³ | | | | | |
| OLC² | Vehicle | −12.093 | 7.327 | 0.397 | −34.626 | 10.441 |
| | Tenapanor³ | −3.583 | 3.884 | 0.793 | −14.299 | 7.134 |
| | OLC² + | 15.961 | 3.647 | <0.001 | 6.216 | 25.706 |
| | Tenapanor³ | | | | | |
| OLC² + | Vehicle | −28.053 | 7.090 | 0.016 | −50.409 | −5.698 |
| Tenapanor³ | Tenapanor³ | −19.543 | 3.417 | <0.001 | −29.195 | −9.892 |
| | OLC² | −15.961 | 3.647 | <0.001 | −25.706 | −6.216 |

¹Mean Urinary phosphate excretion levels for each rat from Days 9 to 11 were averaged by treatment group
²Average of all OLC doses (0.75%, 1.5%, 3%) in chow
³Tenapanor dose = 0.15 mg/kg/po/bid

The Bliss model of independence was employed to determine if the combination of OLC and tenapanor was independent, synergistic, or antagonistic. (Zhao W, et al. J Biomol Screen June 2014; 19 (5): 817-821). The Bliss model compares the predicted combination response, calculated using the complete additivity of probability theory based on the observed effect of each individual agent administered alone, with the observed effect of combination treatment.

The mean percentage reduction in urinary phosphate excretion relative to vehicle by treatment group was calculated by averaging urinary phosphate excretion for each animal from Days 9 to 11. Paired t-tests were conducted to assess the statistical significance between observed and predicted results. 2-tailed p-values<0.05 are considered to be statistically significant.

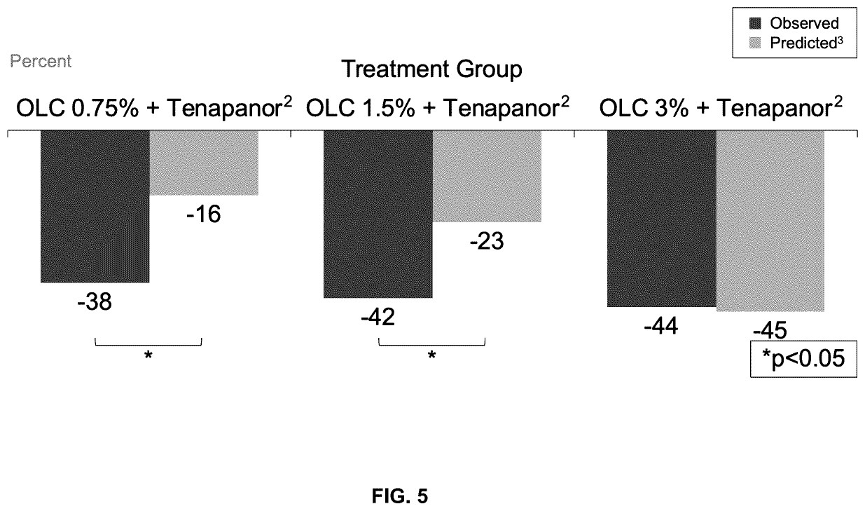

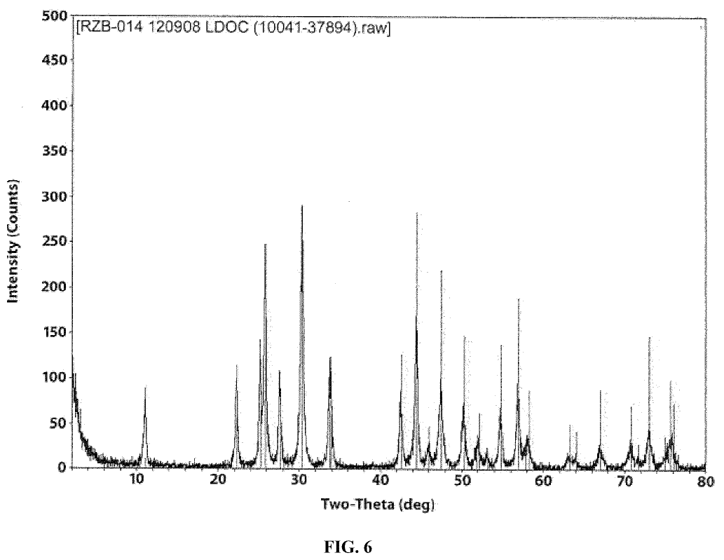

The observed reduction in urinary phosphate excretion for the OLC+tenapanor groups (0.75% and 1.5% OLC doses) was significantly larger than the predicted reduction based on the single-agent effects in accordance with the Bliss model (p=0.009 for 0.75% OLC+tenapanor and p=0.010 for 1.5% OLC+tenapanor) (FIG. 5). The difference in observed (38%) vs. predicted (16%) urinary phosphate excretion was the most pronounced for the combination group with the lowest OLC dose (0.75%). Sizeable reductions were demonstrated in urinary phosphate excretion in response to OLC monotherapy and the most pronounced reductions in urinary phosphate excretion when using OLC in combination with tenapanor. The Bliss model of independence assessed the statistical significance between observed and predicted results and indicated that combination OLC+tenapanor was synergistic (p=0.009 for 0.75% OLC+tenapanor and p=0.010 for 1.5% OLC+tenapanor).

1. A method of reducing serum phosphate in a patient with end-stage renal disease (ESRD) in need thereof, the method comprising orally administering to the patient an oxylanthanum carbonate (OLC) and tenapanor.

2. A method of reducing serum phosphate in a patient with diabetic or non-diabetic chronic kidney disease (CKD) in need thereof, the method comprising orally administering to the patient an oxylanthanum carbonate (OLC) and tenapanor.

3-6. (canceled)

7. A method of reducing the incidence and/or severity of diarrhea associated with administration of tenapanor in a patient suffering from ESRD in need of tenapanor treatment comprising orally administering to the patient an oxylanthanum carbonate (OLC) and tenapanor.

8-11. (canceled)

12. The method of claim 1, wherein upon administration of the oxylanthanum carbonate (OLC) and the tenapanor to the patient, the incidence and/or severity of diarrhea is lower compared to administration to the patient of 30 mg of tenapanor BID.

13-18. (canceled)

19. The method of claim 1, wherein the tenapanor and the OLC are administered in synergistically effective amounts.

20. The method of claim 1, wherein the OLC is administered at a dose of 1000 mg once daily (QD), twice daily (BID) or three times daily (TID).

21. The method of claim 1, wherein OLC is administered at a dose of 500 mg QD, BID or TID.

22. The method of claim 1, wherein the OLC is administered at a dose between 100 mg and 1000 mg QD, BID or TID.

23-28. (canceled)

29. The method of claim 22, wherein the OLC is administered TID.

30. The method of claim 1, wherein the tenapanor is administered at a dose of 30 mg QD, BID or TID.

31. The method of claim 1, wherein the tenapanor is administered at a dose lower than 30 mg QD, BID or TID.

32. (canceled)

33. The method of claim 1, wherein the tenapanor is administered at a dose between 1 mg and 25 mg QD, BID or TID.

34. (canceled)

35. The method of claim 1, wherein the tenapanor is administered at a dose of 0.1 mg, 0.3 mg, 0.5 mg, 1 mg, 2 mg, 3 mg, 4 mg, 5 mg, 10 mg, 15 mg or 20 mg QD, BID or TID.

36. (canceled)

37. The method of claim 1, wherein the tenapanor is administered BID.

38-44. (canceled)

45. The method of claim 1, wherein the method comprises administering the OLC and tenapanor by swallowing an intact dosage form containing the tenapanor and an intact dosage form containing the OLC.

46. The method of claim 1, wherein the method comprises administering the OLC and tenapanor by swallowing an intact dosage form containing the tenapanor and the OLC.

47. The method of claim 1, wherein the administration of the OLC and tenapanor together result in a serum phosphate level in the patient that is lower compared to a serum phosphate level in a patient administered OLC or tenapanor alone.

48. (canceled)

49. The method of claim 47 wherein the administration of the OLC and tenapanor together result in a serum phosphate level in the patient that is between 10% and 50% lower compared to a serum phosphate level in a patient administered OLC or tenapanor alone.

50. A dosage form for oral administration comprising an effective amount of an oxylanthanum carbonate (OLC) and tenapanor.

51-80. (canceled)
